(19) United States
(12) Patent Application Publication
(10) Pub. No.: US 2014/0072624 A1
(43) Pub. Date: Mar. 13, 2014

(54) UNIT DOSE FORM FOR ORAL ADMINISTRATION

(71) Applicants: Donald Jung, South San Francisco, CA (US); Mark Matteucci, South San Francisco, CA (US); Stewart Kroll, South San Francisco, CA (US)

(72) Inventors: Donald Jung, South San Francisco, CA (US); Mark Matteucci, South San Francisco, CA (US); Stewart Kroll, South San Francisco, CA (US)

(73) Assignee: THRESHOLD PHARMACEUTICALS, INC., South San Francisco, CA (US)

(21) Appl. No.: 14/110,819

(22) PCT Filed: Apr. 13, 2012
(86) PCT No.: PCT/US2012/033671

§ 371 Date: Oct. 31, 2013

Related U.S. Application Data
(60) Provisional application No. 61/475,844, filed on Apr. 15, 2011.

(51) Int. Cl. A61K 31/675; A61K 9/20; A61K 9/50

(52) U.S. Cl. CPC A61K 31/675; A61K 9/5047; A61K 9/2081
USPC 424/451; 548/119; 424/495; 424/464; 514/94

(57) ABSTRACT

Formulations and unit dose forms of TH-302 and other hypoxia activated prodrugs suitable for oral administration are useful for treating cancer.

### CROSS-REFERENCE TO RELATED APPLICATION

This application claims priority under 35 U.S.C. 119(e) of U.S. provisional application No. 61/475,844 filed on Apr. 15, 2011, the contents of which are incorporated herein by reference.

### FIELD OF THE INVENTION

The present invention relates to pharmaceutical formulations and unit dose forms of hypoxia activated prodrugs suitable for oral administration to treat cancer and methods of treating cancer by administering such formulations and unit dose forms. The invention thus relates to the fields of medicine and pharmacology.

### BACKGROUND OF THE INVENTION

TH-302 is a hypoxia activated prodrug in clinical testing for treatment of cancer, where it is administered as an intravenous (IV) infusion and has shown excellent clinical activity. TH-302 is a nitroimidazole-linked prodrug of a brominated version (Br-IPM) of isophosphoramide mustard (IPM) with the following structure (the conversion of TH-302 to Br-IPM takes place under hypoxic conditions):

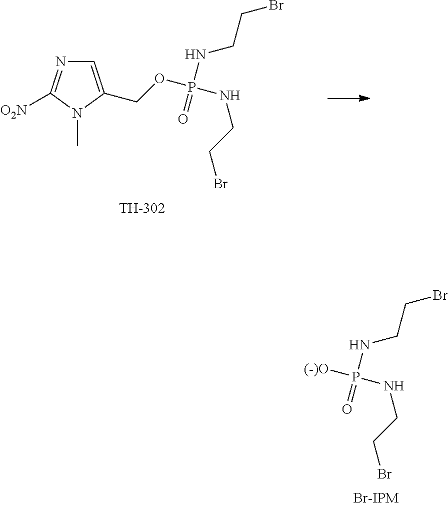

Methods for TH-302's synthesis and use are described in PCT Pub. Nos. WO 2007/002931; WO 2008/083101; WO 2010/048330; WO 2012/006032; and WO 2012/009288; and PCT patent application No. PCT/US2012/031677, each of which is incorporated herein by reference.

There is a need for oral formulations of TH-302 and other hypoxia activated prodrugs; this invention meets that need.

### SUMMARY OF THE INVENTION

In one aspect, the present invention provides an oral formulation, preferably a modified release formulation, comprising or consisting essentially of TH-302, or another hypoxia activated prodrug of Formula I (see below), and optionally one or more pharmaceutically acceptable excipients. As used herein, the excipient is suitable for administration to human cancer patients. In some embodiments, the formulation is a solid formulation, which in some embodiments is provided in a unit dose form such as a tablet or a capsule. In other embodiments, the formulation is a liquid formulation.

Though oral administration offers advantages such as lower hospitalization costs and easier administration, anti-cancer agents such as TH-302 are typically administered by IV infusion, due to their toxicity, size, and/or generally poor oral bioavailability. TH-302 is a prodrug, and its toxicity is substantially masked until the toxin it contains is unmasked, e.g., under hypoxia. Moreover, as demonstrated by the studies disclosed herein, TH-302 has surprisingly high bioavailability. Therefore, the oral formulations of TH-302 provided by the present invention offer certain advantages over its liquid formulations suitable for IV infusion. Given TH-302's half life, the modified release oral formulations of TH-302 provided by the present invention also have therapeutically beneficial applications. Accordingly, oral formulations of other hypoxia activated prodrugs, as provided herein, are also therapeutically beneficial.

In various embodiments, the hypoxia activated prodrug present in the formulations and unit dose forms of the invention is a compound of Formula I:

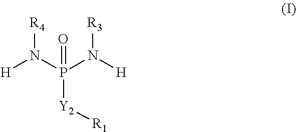

wherein Y₂ is O, S, NR₆, NCOR₆, or NSO₂R₆ wherein R₆ is C₁-C₆ alkyl, C₁-C₆ heteroalkyl, aryl, or heteroaryl; R₃ and R₄ are independently selected from the group consisting of 2-haloalkyl, 2-alkylsulfonyloxyalkyl, 2-heteroalkylsulfonyloxyalkyl, 2-arylsulfonyloxyalkyl, and 2-heteroalkylsulfonyloxyalkyl; R₁ has the formula L-Z₃; L is C(Z₁)₂; each Z₁ independently is hydrogen, halogen, C₁-C₆ alkyl, C₁-C₆ heteroalkyl, aryl, heteroaryl, C₃-C₈ cycloalkyl, heterocyclyl, C₁-C₆ acyl, C₁-C₆ heteroacyl, aroyl, or heteroaroyl; or L is:

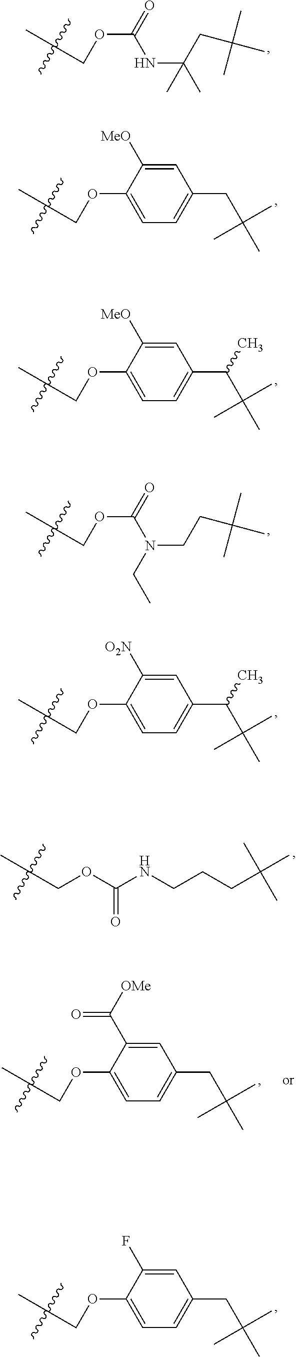

Z₃ is a bioreductive group having a formula selected from the group consisting of:

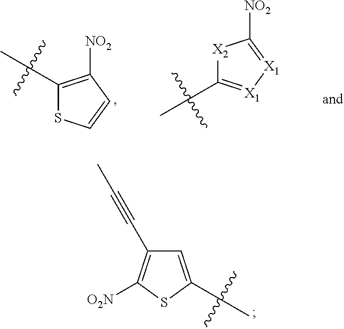

wherein each X₁ is independently N or CR₈; X₂ is NR₇, S, or O; each R₇ is independently C₁-C₆ alkyl, C₁-C₆ heteroalkyl, C₃-C₈ cycloalkyl, heterocyclyl, aryl or heteroaryl; and R₈ is independently hydrogen, halogen, cyano, CHF₂, CF₃, CO₂H, amino, C₁-C₆ alkyl, C₁-C₆ heteroalkyl, C₁-C₆ cycloalkyl, C₁-C₆ alkoxy, C₁-C₆ alkylamino, C₁-C₆ dialkylamino, aryl, CON(R₇)₂, C₁-C₆ acyl, C₁-C₆ heteroacyl, aroyl or heteroaroyl; or a pharmaceutically acceptable salt thereof. In various embodiments, the compound utilized in the oral formulations and methods of this invention is a compound of Formula I selected from the group consisting of TH-281, TH-302, and TH-308 (structures provided below).

In one embodiment, the hypoxia activated prodrug is TH-302.

In another embodiment, the oral formulation is an immediate release formulation, including, without limitation, a tablet or capsule formulation comprising TH-302 or another hypoxia activated prodrug of Formula I.

In another embodiment, the oral formulation is a modified release formulation. In one embodiment, the modified release formulation is a tablet or capsule. In one embodiment, the modified release formulation comprises or consists essentially of microparticles or nanoparticles comprising or consisting essentially of TH-302, or another hypoxia activated prodrug of Formula I. In one embodiment, the modified release formulation comprises or consists essentially of a controlled release matrix. In one embodiment, the modified release formulation comprises a core comprising or consisting essentially of TH-302, or another hypoxia activated prodrug of Formula I. In one embodiment, the modified release formulation further comprises a coat. In one embodiment, the coat is a controlled releasing coat. In one embodiment, the coat is a moisture barrier coat. In one embodiment, the modified release formulation further comprises or consists essentially of one or more of the following: an additive that facilitates water penetrating into the formulation (i.e., the unit dose form containing such formulation), a binder, a diluent, a glidant, a lubricant, a plasticizer, a solubilizer, and/or a swelling enhancer. In one embodiment, the modified release formulation has a pulsatile release profile for releasing TH-302 or another hypoxia activated prodrug of Formula I.

In another aspect, the present invention provides a method of treating cancer comprising administering a therapeutically effective amount of a formulation or a unit dose form provided herein to a patient in need of such treatment. Such methods include monotherapy and combination therapies including TH-302 or another hypoxia activated prodrug of Formula I. In various embodiments, the methods are similar to those described in the PCT publication Nos. WO 2007/002931; WO 2008/083101; WO 2010/048330; WO 2012/006032; and WO 2012/009288; and PCT patent application No. PCT/US2012/031677, each of which is incorporated herein by reference, with the exception that a suitably equivalent dose of TH-302 or another hypoxia activated prodrug of Formula I, as described herein, is used in place of the IV administered dose of TH-302 described therein.

### BRIEF DESCRIPTION OF THE FIGURE

FIG. 1 graphically shows plasma concentration versus time data for TH-302 in Sprague-Dawley rats following a single IV or oral (PO) dose of TH-302, where the values following the IV doses represent mean (±S.D.) concentrations from three rats at each dose level, and the values following the PO dose are a composite from different sets of animals and represent mean (±S.D.) concentrations from three rats per time point.

### DETAILED DESCRIPTION OF THE INVENTION

This detailed description is divided into sections solely for the convenience of the reader, and disclosure found in a section is applicable and relevant to disclosure in any other section.

### DEFINITIONS

All numerical designations, e.g., pH, temperature, time, concentration, and weight, including ranges, are approximations that typically may be varied (+) or (−) by increments of 0.1, 1.0, 10.0, or 100.0 as appropriate. It is to be understood, although not always explicitly stated, that all numerical designations are preceded by the term “about”.

As used in the specification and claims, the singular form “a”, “an”, and “the” includes plural references unless the context clearly dictates otherwise.

Certain terms related to Formula I are defined below.

“Acyl” refers to —CO-alkyl, wherein alkyl is as defined here.

“Aroyl” refers to —CO-aryl, wherein aryl is as defined here.

“Alkoxy” refers to —O-alkyl, wherein alkyl is as defined here.

“Alkenyl” refers to a linear monovalent hydrocarbon radical or a branched monovalent hydrocarbon radical having the number of carbon atoms indicated in the prefix and containing at least one double bond, but no more than three double bonds. For example, (C₂-C₆)alkenyl includes, ethenyl, propenyl, 1,3-butadienyl and the like. Alkenyl can be optionally substituted with substituents, including for example, deuterium (“D”), hydroxyl, amino, mono or di(C₁-C₆)alkyl amino, halo, C₂-C₆ alkenyl ether, cyano, nitro, ethynyl, C₁-C₆ alkoxy, C₁-C₆ alkylthio, —COOH, —CONH₂, mono- or di(C₁-C₆)alkylcarboxamido, —SO₂NH₂, —OSO₂—(C₁-C₆)alkyl, mono or di(C₁-C₆) alkylsulfonamido, aryl, heteroaryl, alkyl or heteroalkylsulfonyloxy, and aryl or heteroarylsulfonyloxy.

“Alkyl” refers to a linear saturated monovalent hydrocarbon radical or a branched saturated monovalent hydrocarbon radical having the number of carbon atoms indicated in the prefix. (C₁-C₆)alkyl can be optionally substituted with substituents, including for example, deuterium (“D”), hydroxyl, amino, mono or di(C₁-C₆) alkyl amino, halo, C₂-C₆ alkenyl ether, cyano, nitro, ethenyl, ethynyl, C₁-C₆ alkoxy, C₁-C₆ alkylthio, —COOH, —CONH₂, mono- or di(C₁-C₆)alkylcarboxamido, —SO₂NH₂, —OSO₂—(C₁-C₆)alkyl, mono or di(C₁-C₆) alkylsulfonamido, aryl, heteroaryl, alkylsulfonyloxy, heteroalkylsulfonyloxy, arylsulfonyloxy or heteroarylsulfonyloxy.

The prefixes (C₁-C_{qq}), C_{1-qq}, and C₁-C_{qq}, wherein qq is an integer from 2-20, have the same meaning. For example, (C₁-C₆)alkyl, C₁₋₆ alkyl, or C₁-C₆ alkyl includes methyl, ethyl, n-propyl, 2-propyl, n-butyl, 2-butyl, tert-butyl, pentyl, and the like. For each of the definitions herein (e.g., alkyl, alkenyl, alkoxy, etc.), when a prefix is not included to indicate the number of main chain carbon atoms in an alkyl portion, the radical or portion thereof will have six or fewer main chain carbon atoms.

“Alkylamino” or mono-alkylamino refers to —NH-alkyl, wherein alkyl is as defined here.

“Alkynyl” refers to a linear monovalent hydrocarbon radical or a branched monovalent hydrocarbon radical having the number of carbon atoms indicated in the prefix and containing at least one triple bond, but no more than two triple bonds. For example, (C₂-C₆)alkynyl includes, ethynyl, propynyl, and the like. Alkynyl can be optionally substituted with substituents, including for example, deuterium (“D”), hydroxyl, amino, mono or di(C₁-C₆)alkyl amino, halo, C₂-C₆ alkenyl ether, cyano, nitro, ethenyl, C₁-C₆ alkoxy, C₁-C₆ alkylthio, —COOH, —CONH₂, mono- or di(C₁-C₆)alkylcarboxamido, —SO₂NH₂, —OSO₂—(C₁-C₆)alkyl, mono or di(C₁-C₆)alkylsulfonamido, aryl, heteroaryl, alkyl or heteroalkylsulfonyloxy, and aryl or heteroarylsulfonyloxy.

“Aryl” refers to a monovalent monocyclic or bicyclic aromatic hydrocarbon radical of 6 to 10 ring atoms which is substituted independently with one to eight substituents, e.g. one, two, three, four of five substituents selected from deuterium (“D”), alkyl, cycloalkyl, cycloalkylalkyl, halo, nitro, cyano, hydroxyl, alkoxy, amino, acylamino, mono-alkylamino, di-alkylamino, haloalkyl, haloalkoxy, heteroalkyl, COR (where R is hydrogen, alkyl, cycloalkyl, cycloalkyl-alkyl, phenyl or phenylalkyl), —(CR′R″)ₙ—COOR (where n is an integer from 0 to 5, R′ and R″ are independently hydrogen or alkyl, and R is hydrogen, alkyl, cycloalkyl, cycloalkylalkyl, phenyl or phenylalkyl) or —(CR′R″)ₙ—CONR^{x}R^{y} (where n is an integer from 0 to 5, R′ and R″ are independently hydrogen or alkyl, and R^{x} and R^{y} are independently selected from hydrogen, alkyl, cycloalkyl, cycloalkylalkyl, phenyl or phenylalkyl). In one embodiment, R^{x} and R^{y} together is cycloalkyl or heterocyclyl. More specifically the term aryl includes, but is not limited to, phenyl, biphenyl, 1-naphthyl, and 2-naphthyl, and the substituted forms thereof.

“Cycloalkyl” refers to a monovalent cyclic hydrocarbon radical of three to seven ring carbons. The cycloalkyl group can have one or more double bonds and can also be optionally substituted independently with one, two, three or four substituents selected from alkyl, optionally substituted phenyl, or —C(O)R^{z} (where R^{z} is hydrogen, alkyl, haloalkyl, amino, mono-alkylamino, di-alkylamino, hydroxyl, alkoxy, or optionally substituted phenyl). More specifically, the term cycloalkyl includes, for example, cyclopropyl, cyclohexyl, cyclohexenyl, phenylcyclohexyl, 4-carboxycyclohexyl, 2-carboxamidocyclohexenyl, 2-dimethylaminocarbonyl-cyclohexyl, and the like.

“Dialkylamino” or di-alkylamino refers to —N(alkyl)₂, wherein alkyl is as defined here.

“Heteroalkyl” refers to an alkyl radical as defined herein with one, two or three substituents independently selected from cyano, —OR′, —NR^{x}R^{y}, and —S(O)ₚR^{z} (where p is an integer from 0 to 2), with the understanding that the point of attachment of the heteroalkyl radical is through a carbon atom of the heteroalkyl radical. R^{w} is hydrogen, alkyl, cycloalkyl, cycloalkyl-alkyl, aryl, aralkyl, alkoxycarbonyl, aryloxycarbonyl, carboxamido, or mono- or di-alkylcarbamoyl. R^{x} is hydrogen, alkyl, cycloalkyl, cycloalkyl-alkyl, aryl or araalkyl. R^{y} is hydrogen, alkyl, cycloalkyl, cycloalkyl-alkyl, aryl, araalkyl, alkoxycarbonyl, aryloxycarbonyl, carboxamido, mono- or di-alkylcarbamoyl or alkylsulfonyl. R^{z} is hydrogen (provided that p is 0), alkyl, cycloalkyl, cycloalkyl-alkyl, aryl, araalkyl, amino, mono-alkylamino, di-alkylamino, or hydroxyalkyl. Representative examples include, for example, 2-hydroxyethyl, 2,3-dihydroxypropyl, 2-methoxyethyl, benzyloxymethyl, 2-cyanoethyl, and 2-methylsulfonyl-ethyl. For each of the above, R^{w}, R^{x}, R^{y}, and R^{z} can be further substituted by amino, halo, fluoro, alkylamino, di-alkylamino, OH or alkoxy. Additionally, the prefix indicating the number of carbon atoms (e.g., C₁-C₁₀) refers to the total number of carbon atoms in the portion of the heteroalkyl group exclusive of the cyano, —OR^{w}, —NR^{x}R^{y}, or —S(O)ₚR^{z} portions. In one embodiment, R^{x} and R^{y} together is cycloalkyl or heterocyclyl.

“Heteroaryl” refers to a monovalent monocyclic, bicyclic or tricyclic radical of 5 to 12 ring atoms having at least one aromatic ring containing one, two, or three ring heteroatoms selected from N, O, or S, the remaining ring atoms being C, with the understanding that the attachment point of the heteroaryl radical will be on an aromatic ring. The heteroaryl ring is optionally substituted independently with one to eight substituents, preferably one, two, three or four substituents, selected from alkyl, cycloalkyl, cycloalkyl-alkyl, halo, nitro, cyano, hydroxyl, alkoxy, amino, acylamino, mono-alkylamino, di-alkylamino, haloalkyl, haloalkoxy, heteroalkyl, —COR (where R is hydrogen, alkyl, phenyl or phenylalkyl, —(CR′R″)ₙ—COOR (where n is an integer from 0 to 5, R′ and R″ are independently hydrogen or alkyl, and R is hydrogen, alkyl, cycloalkyl, cycloalkyl-alkyl, phenyl or phenylalkyl), or —(CR′R″)ₙ—CONR^{x}R^{y} (where n is an integer from 0 to 5, R′ and R″ are independently hydrogen or alkyl, and R^{x} and R^{y} are, independently of each other, hydrogen, alkyl, cycloalkyl, cycloalkyl-alkyl, phenyl or phenylalkyl). In one embodiment, R^{x} and R^{y} together is cycloalkyl or heterocyclyl. More specifically the term heteroaryl includes, but is not limited to, pyridyl, furanyl, thienyl, thiazolyl, isothiazolyl, triazolyl, imidazolyl, isoxazolyl, pyrrolyl, pyrazolyl, pyridazinyl, pyrimidinyl, benzofuranyl, tetrahydrobenzofuranyl, isobenzofuranyl, benzothiazolyl, benzoisothiazolyl, benzotriazolyl, indolyl, isoindolyl, benzoxazolyl, quinolyl, tetrahydroquinolinyl, isoquinolyl, benzimidazolyl, benzisoxazolyl, benzothienyl, indazolyl, pyrrolopyrymidinyl, indolizinyl, pyrazolopyridinyl, triazolopyridinyl, pyrazolopyrimidinyl, triazolopyrimidinyl, pyrrolotriazinyl, pyrazolotriazinyl, triazolotriazinyl, pyrazolotetrazinyl, hexaaza-indenly, and heptaaza-indenyl and the derivatives thereof. Unless indicated otherwise, the arrangement of the hetero atoms within the ring can be any arrangement allowed by the bonding characteristics of the constituent ring atoms.

“Heterocyclyl” or “cycloheteroalkyl” refers to a saturated or unsaturated non-aromatic cyclic radical of 3 to 8 ring atoms in which one to four ring atoms are heteroatoms selected from O, NR (where R is hydrogen, alkyl, cycloalkyl, cycloalkylalkyl, phenyl or phenylalkyl), P(═O)OR^{w}, or S(O)ₚ (where p is an integer from 0 to 2), the remaining ring atoms being C, wherein one or two C atoms can optionally be replaced by a carbonyl group. The heterocyclyl ring can be optionally substituted independently with one, two, three or four substituents selected from alkyl, aryl, arylalkyl, heteroaryl, heteroarylalkyl, cycloalkyl, cycloalkylalkyl, halo, nitro, cyano, hydroxyl, alkoxy, amino, mono-alkylamino, di-alkylamino, haloalkyl, haloalkoxy, —COR (where R is hydrogen, alkyl, cycloalkyl, cycloalkylalkyl, phenyl or phenylalkyl), —(CR′R″)ₙ—COOR (n is an integer from 0 to 5, R′ and R″ are independently hydrogen or alkyl, and R is hydrogen, alkyl, cycloalkyl, cycloalkylalkyl, phenyl or phenylalkyl), or —(CR′R″)ₙ—CONR^{x}R^{y} (where n is an integer from 0 to 5, R′ and R″ are independently hydrogen or alkyl, R^{x} and R^{y} are, independently of each other, hydrogen, alkyl, cycloalkyl, cycloalkylalkyl, phenyl or phenylalkyl). More specifically the term heterocyclyl includes, but is not limited to, tetrahydropyranyl, N-methylpiperidin-3-yl, N-methylpyrrolidin-3-yl, 2-pyrrolidon-1-yl, pyrrolidinyl, piperidinyl, morpholinyl, tetrahydrofuranyl, tetrahydrothiofuranyl, 1,1-dioxo-hexahydro-1Δ⁶-thiopyran-4-yl, tetrahydroimidazo[4,5-c]pyridinyl, imidazolinyl, piperazinyl, and piperidin-2-yl and the derivatives thereof. The prefix indicating the number of carbon atoms (e.g., C₃-C₁₀) refers to the total number of carbon atoms in the portion of the cycloheteroalkyl or heterocyclyl group exclusive of the number of heteroatoms.

“Heteroacyl” refers to —CO-heteroalkyl, wherein heteroalkyl is as defined here.

“Heteroaroyl” refers to —CO-heteroayl, wherein heteroaryl is as defined here.

“Rₛᵤₗ sulfonyloxy” refers to Rₛᵤₗ—S(═O)₂—O— and includes alkylsulfonyloxy, heteroakylsulfonyloxy, cycloalkylsulfonyloxy, heterocyclylsulfonyloxy, arylsulfonyloxy and heteroarylsulfonyloxy wherein Rₛᵤₗ is alkyl, heteroakyl, cycloalkyl, heterocyclyl, aryl and heteroaryl respectively, and wherein alkyl, heteroakyl, cycloalkyl, heterocyclyl, aryl and heteroaryl are as defined here. Examples of alkylsulfonyloxy include Me-S(═O)₂—O—, Et-S(═O)₂—O—, CF₃—S(═O)₂—O— and the like, and examples of arylsulfonyloxy include:

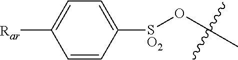

wherein Rₐᵣ is H, methyl, or bromo.

“Substituents” refer to, along with substituents particularly described in the definition of each of the groups above, those selected from: deuterieum, -halogen, —OR′, —NR′R″, —SR′, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NH—C(NH₂)═NH, —NR′C(NH₎═NH, —NH—C(NH₂)═NR′, —S(O)R′, —S(O)₂R′, —S(O)₂NR′R″, —NR′S(O)₂R″, —CN, —NO₂, —R′, —N₃, perfluoro(C₁-C₄)alkoxy, and perfluoro(C₁-C₄)alkyl, in a number ranging from zero to the total number of open valences on the radical; and where R′, R″ and R″ are independently selected from hydrogen, C₁₋₈ alkyl, C₃₋₆ cycloalkyl, C₂₋₈ alkenyl, C₂₋₈ alkynyl, unsubstituted aryl and heteroaryl, (unsubstituted aryl)-C₁-₄ alkyl, and unsubstituted aryloxy-C₁-₄ alkyl, aryl substituted with 1-3 halogens, unsubstituted C₁-₈ alkyl, C₁-₈alkoxy or C₁-₈ thioalkoxy groups, or unsubstituted aryl-C₁-₄ alkyl groups. When R′ and R″ are attached to the same nitrogen atom, they can be combined with the nitrogen atom to form a 3-, 4-, 5-, 6-, or 7-membered ring. For example, —NR′R″ is meant to include 1-pyrrolidinyl and 4-morpholinyl. Other suitable substituents include each of the above aryl substituents attached to a ring atom by an alkylene tether of from 1-4 carbon atoms. Two of the substituents on adjacent atoms of the aryl or heteroaryl ring may optionally be replaced with a substituent of the formula -T²-C(O)—(CH₂)_{q}-U³-, wherein T² and U³ are independently —NH—, —O—, —CH₂— or a single bond, and q is an integer of from 0 to 2. Alternatively, two of the substituents on adjacent atoms of the aryl or heteroaryl ring may optionally be replaced with a substituent of the formula -A-(CH₂₎ᵣ-B-, wherein A and B are independently —CH₂—, —O—, —NH—, —S—, —S(O)—, —S(O)₂—, —S(O)₂NR′— or a single bond, and r is an integer of from 1 to 3. One of the single bonds of the new ring so formed may optionally be replaced with a double bond. Alternatively, two of the substituents on adjacent atoms of the aryl or heteroaryl ring may optionally be replaced with a substituent of the formula —(CH₂)ₛ—X⁵—(CH₂)ₜ—, wherein s and t are independently integers of from 0 to 3, and X⁵ is —O—, —NR′—, —S—, —S(O)—, —S(O)₂—, or —S(O)₂NR′—. The substituent R′ in —NR′— and —S(O)₂NR′— is selected from hydrogen or unsubstituted C₁-₆ alkyl.

Certain compounds utilized in the present invention possess asymmetric carbon atoms (optical centers) or double bonds; the racemates, diastereomers, geometric isomers, regioisomers and individual isomers (e.g., separate enantiomers) are all intended to be encompassed within the scope of the present invention. The compounds of the present invention may also contain unnatural proportions of atomic isotopes at one or more of the atoms that constitute such compounds. For example, the compounds may be radiolabeled with radioactive isotopes, such as for example, and without limitation, tritium (³H), iodine-125 (¹²⁵I or carbon-14 (¹⁴C). All isotopic variations of the compounds of the present invention, whether radioactive or not, are intended to be encompassed within the scope of the present invention.

Other terms related to this invention are defined below.

“Administering” or “administration of” a drug to a patient (and grammatical equivalents of this phrase) refers to direct administration, which may be administration to a patient by a medical professional or may be self-administration, and/or indirect administration, which may be the act of prescribing a drug. For example, a physician who instructs a patient to self-administer a drug and/or provides a patient with a prescription for a drug is administering the drug to the patient.

“Cancer” refers to malignant solid tumors of potentially unlimited growth, as well as various blood cancers that may originate from cancer stem cells in the bone marrow, which can expand locally by invasion and systemically by metastasis. Examples of cancers include, but are not limited to, cancer of the adrenal gland, bone, brain, breast, bronchi, colon and/or rectum, gallbladder, gastrointestinal tract, head and neck, kidneys, larynx, liver, lung, neural tissue, pancreas, prostate, parathyroid, skin, stomach, and thyroid. Other examples of cancers include, adenocarcinoma, adenoma, basal cell carcinoma, cervical dysplasia and in situ carcinoma, Ewing's sarcoma, epidermoid carcinomas, giant cell tumor, glioblastoma multiforma, hairy-cell tumor, intestinal ganglioneuroma, hyperplastic corneal nerve tumor, islet cell carcinoma, Kaposi's sarcoma, leiomyoma, leukemias, lymphomas, malignant carcinoid, malignant melanomas, malignant hypercalcemia, marfanoid habitus tumor, medullary carcinoma, metastatic skin carcinoma, mucosal neuroma, myelodisplastic syndrome, myeloma, mycosis fungoides, neuroblastoma, osteosarcoma, osteogenic and other sarcoma, ovarian tumor, pheochromocytoma, polycythermia vera, primary brain tumor, small-cell lung tumor, squamous cell carcinoma of both ulcerating and papillary type, seminoma, soft tissue sarcoma, retinoblastoma, rhabdomyosarcoma, renal cell tumor or renal cell carcinoma, veticulum cell sarcoma, and Wilm's tumor. Examples of cancers also include astrocytoma, a gastrointestinal stromal tumor (GIST), a glioma or glioblastoma, renal cell carcinoma (RCC), hepatocellular carcinoma (HCC), and pancreatic neuroendocrine cancer. Examples of blood cancers, include, without limitation, acute myelogenous leukemia (AML), acute lymphoblastic leukemia (ALL), chronic myelogenous leukemia (CML), chronic lymphocytic leukemia (CLL), chronic myelomonocytic leukemia (CMML), myelodysplastic syndrome (MDS), myelofibrosis (MF), multiple myeloma (MM), monoclonal gammopathy of undetermined significance (MGUS), follicular lymphoma, mantle cell lymphoma (MCL), diffuse large B cell lymphoma (DLCBL), Burkitt lymphoma, hepatosplenic T cell lymphoma, blastic NK cell lymphoma, cutaneous T cell lymphoma (CTCL), anaplastic large cell lymphoma, and Hodgkin lymphoma.

As used herein, the term “comprising” means any recited elements are necessarily included and other elements may optionally be included. “Consisting essentially of” means any recited elements are necessarily included, elements that would materially affect the basic and novel characteristics of the listed elements are excluded, and other elements may optionally be included. “Consisting of” means that all elements other than those listed are excluded. Embodiments defined by each of these terms are within the scope of this invention.

“Combination therapy” or “combination treatment” refers to the use of two or more drugs in therapy, i.e., use of a hypoxia activated prodrug as described herein together with another anti cancer agent(s), to treat cancer. Examples of such other anti cancer agents include, without limitation, pemetrexed, mTOR inhibitors, HDR inhibitors, gemcitabine, doxorubicin, docetaxel, and angiogenesis inhibitors. Administration in “combination” refers to the administration of two or more agents (e.g., a hypoxia activated prodrug and one or more anti cancer agents, for treating cancer) in any manner in which the pharmacological effects of both are manifest in the patient at the same time. Thus, administration in combination does not require that a single pharmaceutical composition, the same dosage form, or the same route of administration be used for administration of both agents or that the two agents be administered at precisely the same time.

“Control releasing coat” or “controlled release coat” refers to a functional coat which can, for example, include at least one pH independent or pH dependent (such as for example enteric or reverse enteric types) polymer, soluble or insoluble polymers, lipids or lipidic materials, or combinations thereof, which, when applied onto a formulation can slow (for example, when applied to an immediate release formulation or a normal release matrix formulation), further slow (for example when applied to a controlled release matrix formulation), or modify the rate of TH-302, or another hypoxia activated prodrug of Formula I, release.

“Control releasing matrix” or “controlled release matrix” refers to a formulation in which the TH-302, or another hypoxia activated prodrug of Formula I, is included within a matrix, which matrix can be either insoluble, sparingly soluble, soluble, or partly soluble. Controlled release matrix formulations of the insoluble type are also referred to as insoluble polymer matrices, swellable matrices, or lipid matrices, depending on the components that make up the matrix. Controlled release matrix formulations of the soluble type are also referred to as hydrophilic colloid matrices, erodible matrices, or reservoir systems. Controlled release matrix formulations of the present invention refer to formulations comprising an insoluble matrix, a soluble matrix or a combination of insoluble and soluble matrices in which the rate of release is slower than that of an uncoated non-matrix, an immediate release formulations, or an uncoated normal release matrix formulations. Controlled release matrix formulations can be coated with a control releasing coat to further slow the release of TH-302, or another hypoxia activated prodrug of Formula I, from the controlled release matrix formulation. Such coated controlled release matrix formulations can exhibit modified-release, controlled-release, sustained-release, extended-release, prolonged-release, delayed-release, or combinations thereof, of TH-302 or another hypoxia activated prodrug of Formula I.

“Core” refers to a structure that is surrounded by a wall, membrane, or coating. The wall, membrane, or coating can be a functional or non-functional coating.

“Elixir” refers to a liquid formulation including, without limitation, a syrup, glycerine, or alcohol added, e.g. to mask an active agent's unpleasant taste.

“Enhanced absorption formulation” refers to a formulation that demonstrates enhanced absorption of TH-302, or another hypoxia activated prodrug of Formula I, such that, when exposed to like conditions, it will show higher release and/or more absorption of TH-302, or another hypoxia activated prodrug of Formula I, as compared to an immediate release formulation with the same or higher amount of TH-302, or another hypoxia activated prodrug of Formula I, that does not contain the excipients that provide for enhanced absorption. For example, and without limitation, the same therapeutic effect can be achieved with less TH-302, or another hypoxia activated prodrug of Formula I, in an enhanced absorption formulation as compared to the corresponding immediate release form.

“Enteric coating” refers to a barrier applied to oral formulations that controls the location in the digestive system where the contents of the oral formulation is absorbed. For example, enteric coatings prevent release of the contents before they reach the small intestine.

“Enteric polymer” refers to a polymer presenting a surface that is stable at the acidic stomach pH, but breaks down at a less acidic (relatively more basic) pH. For example, such a polymer will not dissolve, or dissolve only partially, in the acidic juices of the stomach (pH ˜3), but they will, in the alkaline (pH 7-9) environment present in the small intestine. Enteric polymers are suitable for use in moisture barriers and/or enteric coatings.

“Excipient” refers to a pharmacologically inactive, pharmaceutically acceptable substance used with the active agents or drugs of a formulation. Excipients are also used to bulk up formulations that contain very potent active ingredients, to allow for convenient and accurate dosage in the unit dose form. In addition to their use in the unit dose forms, excipients can be used in the manufacturing process to aid in the handling of the active substance concerned. Depending on the route of administration, and form of medication, different excipients may be used. Examples of excipients include, without limitation, one or more of the following: an additive, an anti-foaming agent, a binder, a chemical stabilizer, a coloring agent, a diluent, a disintegrating agent, an emulsifying agent, a filler, a flavoring agents, a glidant, a lubricant, a pH modifier, a plasticizer, a solubilizer, a swelling enhancer, a spheronization aid, a solubility enhancer, and a suspending agent.

“Extended or sustained release formulation” refers to a formulation that demonstrates extended or sustained release of TH-302, or another hypoxia activated prodrug of Formula I, relative to an immediate release formulation, such that, for example and without limitation, when administered once or twice daily, the formulation releases TH-302, or another hypoxia activated prodrug of Formula I, slowly, so that plasma concentrations of TH-302, or another hypoxia activated prodrug of Formula I, are maintained at a therapeutic level for an extended period of time.

“Hypoxia activated prodrug” refers to a drug that is less active or inactive under normoxia than under hypoxia or anoxia. Hypoxia activated prodrugs include drugs that are activated, e.g., by a variety of reducing agents and reducing enzymes, including without limitation single electron transferring enzymes (such as cytochrome P450 reductases) and two electron transferring (or hydride transferring) enzymes (see U.S. Pat. App. Pub. Nos. 2005/0256191, 2007/0032455, and 2009/0136521, and PCT Pub. Nos. 2000/064864, 2004/087075, and 2007/002931, each of which is incorporated herein by reference). The hypoxia activated prodrugs useful in the oral formulations and methods of the present invention include compounds of Formula I, including but not limited to compounds where Z₃, as defined by that formula, is a 2-nitroimidazole moiety. Examples of particular hypoxia activated prodrugs useful in the methods of the invention include without limitation TH-281, TH-302, and TH-308. Methods of synthesizing, formulating, and using TH-302 and other compounds of Formula I are described in PCT Pub. Nos. WO 2007/002931; WO 2008/083101; WO 2010/048330; WO 2012/006032; and WO 2012/009288; and PCT patent application No. PCT/US2012/031677, each of which is incorporated herein by reference.

“Hyperproliferative disease” refers to a disease characterized by cellular hyperproliferation (e.g., an abnormally increased rate or amount of cellular proliferation). Cancer is a hyperproliferative disease. Examples of hyperproliferative diseases other than cancer include, but are not limited to, allergic angiitis and granulomatosis (Churg-Strauss disease), asbestosis, asthma, atrophic gastritis, benign prostatic hyperplasia, bullous pemphigoid, coeliac disease, chronic bronchitis and chronic obstructive airway disease, chronic sinusitis, Crohn's disease, demyelinating neuropathies, dermatomyositis, eczema including atopic dermatitis, eustachean tube diseases, giant cell arteritis, graft rejection, hypersensitivity pneumonitis, hypersensitivity vasculitis (Henoch-Schonlein purpura), irritant dermatitis, inflammatory hemolytic anemia, inflammatory neutropenia, inflammatory bowel disease, Kawasaki's disease, multiple sclerosis, myocarditis, myositis, nasal polyps, nasolacrimal duct diseases, neoplastic vasculitis, pancreatitis, pemphigus vulgaris, primary glomerulonephritis, psoriasis, periodontal disease, polycystic kidney disease, polyarteritis nodosa, polyangitis overlap syndrome, primary sclerosing cholangitis, rheumatoid arthritis, serum sickness, surgical adhesions, stenosis or restenosis, scleritis, scleroderma, strictures of bile ducts, strictures of duodenum, small bowel, and colon, silicosis and other forms of pneumoconiosis, type I diabetes, ulcerative colitis, ulcerative proctitis, vasculitis associated with connective tissue disorders, vasculitis associated with congenital deficiencies of the complement system, vasculitis of the central nervous system, and Wegener's granulomatosis.

“Immediate release formulation” refers to a formulation from which the drug is released without any substantial delay and substantially at once.

“Microparticle” or “nanoparticle” refers to a drug formulation in discrete particulate form, and is interchangeable with such terms as “microspheres”, “spherical particles”, “microcapsules”, “particles”, “multiparticulates”, “granules”, “spheroids”, “beads”, and “pellets,” though, as will be apparent to the skilled artisan, nanoparticles are typically about 10 folds to about 1000 folds smaller than microparticles.

“Modified release formulation” refers to a formulation with drug release characteristics of time course and/or location that accomplish therapeutic or convenience objectives not offered by immediate release or uncoated normal matrix formulations. See, for example, U.S. Pat. Nos. 6,245,357 and 7,968,120, each of which are incorporated herein by reference. The rate of release of the active drug from a modified release formulation is controlled by features of the formulation and/or in combination with physiologic or environmental conditions rather than by physiologic or environmental conditions alone. In contrast to immediate release, or uncoated normal matrix formulations, which typically produce large differences between maximum and minimum plasma drug concentrations (Cₘₐₓ and Cₘᵢₙ) due to rapid absorption of the drug into the body, the rate of release of the active drug from a modified release formulation produces smaller differences. In immediate release or uncoated normal matrix formulations, the drug content is released into the gastrointestinal tract within a short period of time, and plasma drug levels peak shortly after dosing. The design of immediate release or uncoated normal matrix formulations is generally based on getting the fastest possible rate of drug release. Modified release formulations include those that release the drug (or some portion of the drug in the dosage form) more slowly than an immediate release formulation.

“Moisture barrier” refers to a barrier that impedes or retards the absorption of moisture by a formulation in vivo or under similar conditions. In various embodiments, the moisture barrier is comprised of an enteric and/or acrylic polymer, and optionally may include a plasticizer and/or a permeation enhancer.

“Monotherapy” or “single agent therapy” refers to using a single drug to treat a disease, i.e., using a hypoxia activated prodrug such as, for example, TH-302, or another hypoxia activated prodrug of Formula I, as the only chemical agent to treat cancer. Administration of palliatives and/or vitamins and/or other agents that are administered for purposes other than to directly treat the disease can be administered for monotherapy. A patient undergoing monotherapy may also undergo radiation therapy and/or surgery.

“Patient” or “subject” refers to mammals, particularly humans, and so includes animals of veterinary and research interest, such as simians, cattle, horses, dogs, cats, and rodents with cancer or another hyperproliferative disease.

“Permeation enhancer” refers to a hydrophilic substance, which, when applied as part of a coat on a formulation, allows water to enter the formulation without substantial physical disruption of the coat.

“Pharmaceutically acceptable” refers to a composition that is safe, non-toxic, and is suitable for administration to patients.

“Pharmaceutically acceptable salt” refers to pharmaceutically acceptable salts derived from a variety of organic and inorganic counter ions well known in the art that include, by way of example only, sodium, potassium, calcium, magnesium, ammonium, and tetraalkylammonium, and when the molecule contains a basic functionality, salts of organic or inorganic acids, such as hydrochloride, hydrobromide, tartrate, mesylate, acetate, maleate, and oxalate. Suitable salts include those described in P. Heinrich Stahl, Camille G. Wermuth (Eds.), Handbook of Pharmaceutical Salts Properties, Selection, and Use, 2002, incorporated herein by reference.

“Plasticizer” refers to a compound capable of plasticizing or softening a polymer or a binder. Plasticizers can broaden the average molecular weight of a polymer in which they are included thereby lowering its glass transition temperature or softening point. Plasticizers also can reduce the viscosity of a polymer. Plasticizers can be included in a formulation to modify the properties and characteristics of the polymers used in the coat(s) or core of the formulation for convenient processing during manufacture of the coat(s) and/or the core of the formulation. During manufacture of the coat(s) and/or the core, the plasticizer can lower the melting temperature or glass transition temperature (softening point temperature) of the polymer or binder. Once the coat(s) and/or the core has been manufactured, certain plasticizers can function to increase the hydrophilicity of the coat(s) and/or the core of the formulation in the environment of use. During manufacture of the coat(s) and/or core, the plasticizer can lower the melting temperature or glass transition temperature (softening point temperature) of the polymer or binder.

“Solid formulation” refers to a formulation that is neither liquid nor gaseous. Solid formulations include, without limitation, tablets, powders, particulates such as microparticles and nanoparticles, capsules, matrix forms, suppositories, sachets, troches, patches, and lozenges. Solid formulations in the form of capsules contain a solid composition within a capsule that can be made of gelatin or other encapsulating material. Liquid formulations include, without limitation, liquid suspensions, solutions, and elixirs.

“Swelling enhancer” refers to an excipient that swells rapidly resulting in an increase in the size of a solid formulation, such as a tablet. At lower concentrations, these excipients can be used as super disintegrants; however at higher concentrations, e.g., at concentrations above about 5% w/w, these excipients function as swelling enhancers and increase the size of a matrix formulation.

TH302 or TH-302 refers to the compound of formula:

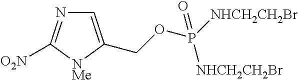

and includes a pharmaceutically acceptable salt thereof.

TH281 or TH-281 refers to the compound of formula:

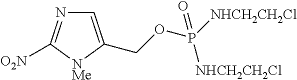

and includes a pharmaceutically acceptable salt thereof.

TH308 or TH-308 refers to the compound of formula:

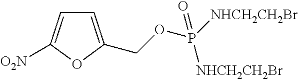

and includes a pharmaceutically acceptable salt thereof.

“Therapeutically effective amount” refers to an amount of the drug that, when administered to a patient with cancer or another hyperproliferative disease, will have the intended therapeutic effect, e.g., alleviation, amelioration, palliation or elimination of one or more manifestations of cancer or other hyperproliferative disease in the patient. A therapeutic effect does not necessarily occur by administration of one dose, and may occur only after administration of a series of doses. Typically, cancer drugs are administered in a repeating series of doses, and in certain instances each series may be referred to as a “cycle” of therapy. Thus, a therapeutically effective amount may be administered in one or more administrations.

“Treating” or “treatment of” a condition or patient refers to taking steps to obtain beneficial or desired results, including clinical results. For purposes of this invention, beneficial or desired clinical results include, but are not limited to, alleviation or amelioration of one or more symptoms of cancer (or other hyperproliferative disease), including conditional survival and reduction of tumor load or volume; diminishment of extent of disease; delay or slowing of disease progression; amelioration, palliation, or stabilization of the disease state; or other beneficial results.

### Oral Formulations of TH-302 and Other Hypoxia Activated Prodrugs

When male Sprague-Dawley rats were administered a single oral dose of 100 mg/kg of TH-302 in normal saline, TH-302 was absorbed rapidly, and the mean peak plasma concentration (15.8 μg/mL) was reached by the first (0.25 hours) sampling time point. The corresponding mean area under the concentration curve (AUC) and half-life were 13.1 μg-h/mL and 1.6 h. Following the administration of the same amount (100 mg/kg) by a 30 minute intravenous (IV) infusion, the mean peak plasma concentration at the end of infusion was 30.9 μg/mL with a mean AUC and half-life of 17.1 μg-h/mL and 0.27 h, respectively. Based on these two tests, the absolute bioavailability of TH-302 was determined to be approximately 77%, demonstrating that TH-302 is suitable for oral administration.

TH-302, prior to the present invention, has been administered to humans intravenously (IV), in doses of about 100 mg/m² (where “m²” refers to the body surface area of the patient to be treated) to about 700 mg/m², such as 240 mg/m² or 340 mg/m², or 575 mg/m². Typically, such doses were often administered on a once-weekly regimen. It has also been administered at a dose of about 450 mg/m², 5 days a week, for example, to advanced leukemia patients. For compliance reasons, an optimal oral dose of an immediate release formulation of the present invention will be administered once (QD), twice (BID), or three times per day (TID), and continuous daily dosing will be employed in many settings. With such continuous daily dosing administration, the oral daily dose will, in general, be in the range of about one fourth to about one half of the weekly dose, i.e., about one third of the weekly dose. Given the less than 100% oral bioavailability in humans that can be predicted (e.g. about 70%-about 80% oral bioavailability in humans) from the rat based tests described herein, the oral daily dose is contemplated to be, for example, about 50 mg/m²-about 900 mg/m², about 100 mg/m²-about 800 mg/m², about 200 mg/m²-about 700 mg/m², about 50 mg/m²-about 200 mg/m², or about 700 mg/m²-about 900 mg/m², depending on the nature of the cancer to be treated and whether additional chemotherapeutic agents are co-administered (typically, lower doses of TH-302 will be used in a combination therapy as opposed to TH-302 monotherapy). In one illustrative example, the oral daily dose is about 150 mg/m².

Given the typical adult human body surface area (BSA) of about 1.73 m², the typical daily dose of an oral formulation of the invention will range from about 100 mg-about 1600 mg, about 200 mg-about 1500 mg, about 300 mg-about 1400 mg, about 100 mg-about 200 mg, or about 1200 mg-about 1600 mg.

In one embodiment, the daily dose of an oral formulation, particularly, that of TH-302, is about 520 mg-about 590 mg, such as 500 mg, 550 mg, or 600 mg. In one embodiment, the daily dose of an oral formulation is about 735 mg-about 840 mg, such as 700 mg, 750 mg, 800 mg, or 850 mg. Such a daily dose is particularly suited for once weekly administration. Lower amounts, such as one fifth (⅕) or one seventh ( 1/7) of these amounts are suitable for a administering the hypoxia activated prodrug every day. Such lower daily amounts include 100 mg-170 mg amounts.

As will be apparent from this disclosure, however, the present invention provides a variety of formulations and unit dose forms and dosing regimens for orally administered TH-302, or another hypoxia activated prodrug, therapy. Modified release formulations, including without limitation, sustained and pulsatile release formulations, are provided, for example, that permit QD administration as an equivalent for any BID or TID administration of an immediate release formulation. Similarly, dosing regimens other than continuous daily administration are provided and include, for example, daily×5 days and daily×3 days administration (on a weekly or three weeks on therapy followed by one week off therapy, for example) and once per week administration.

Though oral administration offers advantages such as lower hospitalization costs and easier administration, anti cancer agents such as TH-302 are typically administered by IV infusion, due to their toxicity, size, and generally poor bioavailability. TH-302 and other compounds of Formula I are prodrugs, and their toxicity is substantially masked until the toxin is unmasked, e.g., under hypoxia. Moreover, as demonstrated by the studies disclosed herein, TH-302 and other compounds of Formula I have surprisingly high bioavailability. Therefore, the oral formulations of TH-302 and other compounds of Formula I provided by the present invention offer certain advantages over its liquid formulations suitable for IV infusion. Given the half life of TH-302 and other compounds of Formula I, the modified release oral formulations of TH-302 provided by the present invention also have therapeutically beneficial application.

In one aspect, the present invention provides an oral formulation comprising or consisting essentially of TH-302, or another hypoxia activated prodrug of Formula I, and optionally an excipient. As used herein, the excipient is suitable for administration to cancer patients, which are typically human patients, although the formulations and dosage forms of the invention have veterinary application as well. In certain embodiments, oral formulations of hypoxia activated drugs of Formula I other than TH-302 are provided. These embodiments can be viewed as the TH-302 formulations specifically described herein in which the TH-302 has been replaced by the other hypoxia activated prodrug of Formula I. In one embodiment, the oral formulation is a solid formulation.

In one embodiment, the oral formulation is an immediate release formulation. Such embodiments include, without limitation, a gelatin capsule or tablet formulation comprising or consisting essentially of TH-302 or another hypoxia activated prodrug of Formula I.

In one embodiment, the oral formulation is a modified release formulation. In one embodiment, the modified release formulation is a tablet. In one embodiment, the modified release formulation comprises microparticles or nanoparticles comprising or consisting essentially of TH-302, or another hypoxia activated prodrug of Formula I. In one embodiment, the modified release formulation comprises a controlled release matrix. In one embodiment, the modified release formulation comprises a core comprising or consisting essentially of TH-302 or another hypoxia activated prodrug of Formula I. In one embodiment, the modified release formulation includes an immediate release component. In one embodiment, the modified release formulation further comprises a coat. In one embodiment, the coat is a controlled releasing coat. In one embodiment, the coat is a moisture barrier coat. In one embodiment, the modified release formulation further comprises an additive selected from the group consisting of additives that facilitate water penetrating into the formulation, a binder, a diluent, a glidant, a lubricant, a plasticizer, a solubilizer, and/or a swelling enhancer. In one embodiment, the modified release formulation has a pulsatile release profile of releasing TH-302 or another hypoxia activated prodrug of Formula I.

### Immediate Release Formulation

In one embodiment, the formulation of the invention is an immediate release formulation comprising or consisting essentially of TH-302 or another hypoxia activated prodrug of Formula I. In one embodiment, the immediate release formulation is an uncoated normal matrix formulation. In one embodiment, the immediate release formulation is a gelatin capsule or tablet containing TH-302 or another hypoxia activated prodrug of Formula I. The TH-302 or other hypoxia activated prodrug contained in the gelatin capsule is, in various embodiments, a powder, a granular substance, or substantially spherical microparticles. Gelatin capsules of the present invention can be manufactured by adapting the methods reported for preparing gelatin capsules for a different type of an anti cancer alkylator, ifosfamide. See Manegold et al., Ann Oncol. 1996; 7(6): 637-9, incorporated herein by reference. An immediate release formulation generally provides a fast rate of drug release and/or a high Cₘₐₓ. Various methods of making other immediate release formulations, which can be applied to making those of the present invention in view of the disclosure herein, are well known to the skilled artisan and can be employed or adapted to make the formulations and dosage forms of the present invention.

### Modified Release Formulation

In one embodiment, the formulation of the invention is a modified release formulation. In one embodiment, the modified release formulation is a monolithic formulation. As used herein, a monolithic formulation refers to a single unit or a tablet, as opposed to multiparticulate formulations. In one embodiment, the modified release formulation is a multiparticulate formulation. Examples of modified release formulations are disclosed in U.S. Pat. Nos. 5,591,452 and 5,965,161, and such examples can be modified in accordance with the present disclosure to prepare modified release formulations of the present invention. In one embodiment, the modified release formulation is coated. In one embodiment, the coating is a functional coating. In one embodiment, the functional coating includes one or more of the following: a polymeric coating, a moisture barrier coating, an enteric polymeric coating, and mixtures of the same. In another embodiment, the coating is a non functional coating. The non functional coating may not substantially affect release of TH-302 or another hypoxia activated prodrug of Formula I, but may instead affect other properties of the formulation, including, without limitation, enhancing chemical, biological, or physical stability of the formulation or dosage form.

### Polymeric Controlled Releasing Coat

In one embodiment, the functional coating is a polymeric coating. Generally, reactive amine containing polymers are avoided in polymeric coatings used for TH-302. In one embodiment, the polymeric coating is a control releasing coating. In one embodiment, the control releasing coating comprises an acrylic polymer. Suitable acrylic polymers include, but are not limited to, acrylic acid and methacrylic acid copolymers, cynaoethyl methacrylate, ethoxyethyl methacrylates, glycidyl methacrylate copolymers, methyl methacrylate copolymers, poly(acrylic acid), polyacrylamide, poly(methacrylic acid), poly(methyl methacrylate), and poly(methacrylic acid anhydride).

In one embodiment, the acrylic polymer is a polymerizable quaternary ammonium compound. Nonlimiting examples of such polymerizable quaternary ammonium compounds include quaternized aminoalkyl esters and aminoalkyl amides of acrylic acid and methacrylic acid, for example β-methacryl-oxyethyl-trimethyl-ammonium methosulfate, β-acryloxy-propyl-trimethyl-ammonium chloride, and trimethylaminomethyl-methacrylamide methosulfate. The quaternary ammonium can also be part of a heterocycle, as in methacryloxyethylmethyl-morpholiniom chloride or the corresponding piperidinium salt, or it can be joined to an acrylic acid group or a methacrylic acid group by way of a group containing hetero atoms, such as a polyglycol ether group. Other suitable polymerizable quaternary ammonium compounds include, without limitation, quaternized vinyl-substituted nitrogen heterocycles such as methyl-vinyl pyridinium salts, vinyl esters of quaternized amino carboxylic acids, styryltrialkyl ammonium salts, and the like. Still other polymerizable quaternary ammonium compounds include, without limitation, acryl- and methacryl-oxyethyltrimethyl-ammonium chloride, benzyldimethylammoniumethyl-methacrylate chloride, diethylmethylammoniumethyl-acrylate, N-trimethylammoniumpropylmethacrylamide chloride, and N-trimethylammonium-2,2-dimethylpropyl-1-methacrylate chloride.

In one embodiment, the polymeric control releasing coating comprises or consists essentially of an acrylic polymer and a polymerizable quaternary ammonium compound.

In one embodiment, the control releasing coat further includes a polymer whose permeability is pH dependent, such as anionic polymers synthesized from methacrylic acid and methacrylic acid methyl ester. In some embodiments, the polymer is insoluble and water impermeable in acids and pure water, but becomes increasingly water permeable above pH 5.0-pH 7.0. Such hydrophobic acrylic polymer can include a cationic polymer based on dimethylaminoethyl methacrylate and neutral methacrylic acid. The hydrophobic acrylic polymer coatings utilized in the present invention can include a neutral copolymer based on poly(meth)acrylates or lacquer films that are insoluble in water and digestive fluids, but are water permeable and water swellable.

In one embodiment, the control releasing coat comprises or consists essentially of polyvinyl acetate stabilized with polyvinylpyrrolidone and sodium lauryl sulfate. The dissolution profile of such a coat can by altered by changing the relative amounts of different acrylic resin lacquers included in the coating. Also, by changing the molar ratio of polymerizable permeability-enhancing agent (e.g., the quaternary ammonium compounds) to the neutral (meth)acrylic esters, the permeability properties (and thus the dissolution profile) of the resultant coating can be modified, as is well known to the skilled artisan.

Other examples of polymers that can be used in the control releasing coat include one or more of agar (including, e.g. a swellable mixture of agar and a cellulose, such as carboxymethyl cellulose); alginates (e.g. ammonia, calcium, sodium, potassium alginates, and propylene glycol alginates), carboxyvinyl polymers, casein, celluloses (i.e., calcium carboxymethyl cellulose, carboxymethyl cellulose, cellulose acetate, cellulose acetate phthalate, cellulose acetate trimaletate, cellulose based cross-linked polymers in which the degree of crosslinking is low so as to facilitate adsorption of water and expansion of the polymer matrix, cellulose butyrate, cellulose ethers, cellulose propionate, chitin, collagen, copolymers of maleic anhydride and styrene, ethylene, propylene, and isobutylene, crosslinked polyvinyl alcohol and poly N-vinyl-2-pyrrolidone (polyvinylpyrrolidone or PVP), diesters of polyglucan, ethyl cellulose, ethylhydroxy ethylcellulose, gelatin, glycerol fatty acid esters, gums (e.g. arabic, karaya, locust bean, tragacanth, carrageens, guar, xanthan, scleroglucan and mixtures and blends thereof), hydroxyethyl cellulose, hydroxypropyl cellulose, hydroxypropyl methylcellulose, hydroxypropyl methylcellulose phthalate, hydrogels (including anionic and cationic hydrogels) and gel-forming materials, hydrophilic polymers, lecithins, maltodextrin, methyl cellulose, methyl ethyl cellulose, microcrystalline cellulose, nitro cellulose, pectin (e.g. mol. wt. 30-300 kD), PVP including those of mol. wt. in the range of 10-360 kD, polyacrylamides, polyacrylic acid, polyamides, polyethylene oxides (e.g. mol. wt. 100 k to 5000 k), poly(hydroxyalkyl methacrylate) (e.g. mol. wt. 5-5000 kD), polysaccharides (e.g. acacia and algins), polyvinyl acetate, polyvinyl alcohol (PVA, including a PVA with low acetate residual, pullulan, polyvinyl acetate phthalate, polyvinyl alcohol phthalate, shellac, sodium carboxymethyl cellulose, starch, including crosslinked starch, swellable hydrophilic polymers, and zein. Other polymers include sodium alginate, sodium carmellose, calcium carmellose, and sodium carboxymethyl starch.

In various embodiments, the formulation of TH-302 or another hypoxia activated prodrug of Formula I is coated with a polymer to facilitate mucoadhesion within the gastrointestinal tract. Non limiting examples of polymers that can be used for mucoadhesion include carboxymethylcellulose, polyacrylic acid, gelatin and other natural or synthetic polymers.

### Tablets

In one embodiment, the modified release formulation of TH-302, or another hypoxia activated prodrug of Formula I, is provided in a unit dose form as a tablet. In one embodiment, the tablet comprises a core comprising TH-302, or another hypoxia activated prodrug of Formula I, and an excipient. When mixed with such an excipient, the core may be an immediate release formulation. In one embodiment, the core is surrounded by a control releasing coat which controls the release of TH-302 or another hypoxia activated prodrug of Formula I. In various embodiments, a moisture barrier surrounds the control releasing coat. If present, the moisture barrier coat retards moisture from coming into contact with the TH-302 or other hypoxia activated prodrug of Formula I. Optionally, this tablet may further comprise or consist essentially of one or more additional functional or non functional coatings surrounding the core, moisture barrier and/or control releasing coat.

### Extended Release Tablets

In one embodiment, the tablet provided by the invention is an extended-release tablet. In one embodiment, the tablet comprises a core comprising TH-302, or another hypoxia activated prodrug of Formula I, and one or more excipients. In one embodiment, the core is surrounded by a control releasing coat, which controls the release of the TH-302 or other hypoxia activated prodrug of Formula I. The tablet may optionally comprise one or more additional functional or non functional coats surrounding the core or control releasing coat.

In one embodiment, the core of the extended-release tablet comprises TH-302, or another hypoxia activated prodrug of Formula I, a binder, and a lubricant and optionally contains other inert excipients. Various binders, lubricants, glidants, and other inert excipients useful in accordance with the present formulations are well known to the skilled artisan and can be readily selected in view of the present disclosure. Additional inert excipients well known to the skilled artisan are found in the relevant literature, for example in the Handbook of Pharmaceutical Excipients, 5^{th} Edition, Edited by Raymond C. Rowe et al., incorporated herein by reference. In some embodiments, the core of the extended release formulation is an uncoated immediate release formulation or a normal release matrix formulation.

These and other tablet cores utilized according to the present disclosure can be manufactured by wet and dry granulation, direct compression, extrusion, spheronization, melt granulation, and rotary granulation processes that are well known to the skilled artisan.

### Coatings

In one embodiment, the tablet cores are coated with an extended release, control releasing coating. In one embodiment, the tablet cores are coated with an aqueous control releasing coating that comprises or consists essentially of an aqueous dispersion of a neutral ester copolymer without any functional groups. In one embodiment, the tablet further comprises a moisture barrier. The control releasing coat and the moisture barrier can be applied in two stages. The control releasing coating can be applied directly onto the surface of the tablet cores and functions primarily to control the release of TH-302 or another hypoxia activated prodrug of Formula I. The moisture barrier can be applied directly onto the surface of the control releasing coat to impede or retard the absorption of moisture by the tablet.

### The Extended Release Control Releasing Coat

In one embodiment, the tablet comprises an extended release control releasing coat. The extended release control releasing coat is a semi permeable coat comprising a water insoluble, water-permeable film-forming polymer, optionally a water-soluble polymer, and still optionally, a plasticizer. Non limiting examples of water-insoluble, water-permeable film-forming polymers useful for the extended release control releasing coat include cellulose ethers, cellulose esters, and polyvinyl alcohol. Other non limiting examples of water-soluble polymers useful for the extended release control releasing coat include, without limitation, hydroxypropyl cellulose, hydroxypropyl methylcellulose, and polyvinylpyrrolidone.

In certain embodiments, the extended release control releasing coat further comprises a plasticizer. Non limiting examples of plasticizers useful in the control releasing coats include without limitation, acetylated monoglycerides, acetyltributyl citrate, acetyltriethyl citrate, butyl phthalyl butyl glycolate, butyl octyl phthalate, castor oil, diacetin, dibenzyl phthalate, dibutyl tartrate, dibutyl phthalate, diethyl phthalate, diethylhexyl phthalate, di-n-octyl phthalate, di-i-octyl phthalate, di-i-decyl phthalate, di-n-undecyl phthalate, di-n-tridecyl phthalate, dihexyl phthalate, dimethyl phthalate, dioctyl azelate, diisononyl phthalate, di-2-ethylhexyl adipate, di-2-ethylhexyl sebacate, di-2-ethylhexyl azelate, dibutyl sebacate, diethyloxalate, diethylmalate, diethylfumerate, dibutylsuccinate, diethylmalonate, dibutylsebacate, epoxidized tallate, ethyl phthalyl ethyl glycolate, glycerin, glycerin sorbitol, gylcerol triacetate, glyceroltributyrate, olive oil, polyethylene glycols, polyhydric alcohols, propylene glycol, rape seed oil, sesame oil, triacetin, tripropioin, triethyl citrate, tri-2-ethylhexyl trimellitate, and triisoctyl trimellitate.

The Moisture Barrier Coat

In certain embodiments, a moisture barrier is in direct contact with the control releasing coat of a unit dose form of the invention. In various embodiments, suitable barriers comprise an enteric polymer, a permeation enhancer, and optionally a plasticizer. In certain embodiments, the enteric polymer is an acrylic polymer. For example, and without limitation, the acrylic polymer can be a methacrylic acid copolymer comprising about 1:1 poly-methacrylic acid-methyl methacrylate.

In one embodiment, the permeation enhancer includes, without limitation, cellulose ethers, hydroxypropylmethylcellulose, and protein-derived materials of these polymers. In another embodiment, the permeation enhancer is cross-linked polyvinylpyrrolidone, polyethylene oxide, polyvinylpyrrolidone, and water-soluble polydextrose, saccharides and polysaccharides, such as pullulan, dextran, sucrose, glucose, lactose, fructose, mannitol, mannose, galactose, sorbitol and the like. Other non limiting examples of permeation enhancers include alkali metal salts such as lithium carbonate, potassium chloride, potassium sulfate, potassium phosphate, sodium acetate, sodium bromide, sodium chloride, sodium citrate, and the like. In some embodiments, the permeation enhancer is a pore forming solid. Examples of pore forming solids include, without limitation, diols, polyols, polyhydric alcohols, polyalkylene glycols, polyglycols, poly(a-w)alkylenediols, and the like. Other permeation enhancers which can be useful in the formulations and unit dose forms of the present invention include starch, modified starch, and starch derivatives, gums, including but not limited to agar, amylose, amylopectin, alginic acid and other alginates, arabinogalactin, biosynthetic gum, carrageenan, kappa-carrageenan, lambda-carrageenan, bentonite, cross-linked polyvinylpyrrolidone, dextran, dextrin, flax seed gum, guar, gum arabic, gum karaya, ion-exchange resins such as potassium polymethacrylate, locust bean gum, okra gum, pectin, quince psyllium, scleroglucan, tragacanth, veegum, and xanthan gum. Still other pore forming solids include materials useful for making microporous lamina in the environment of use, such as acetal polymers, asymmetric porous polymers, collodion, colloidal silica, copolymers or interpolymers having a reduced bulk density, and other similar materials, cross-linked olefin polymers, cross-linked chain-extended poly(urethane), hydrophilic microporous homopolymers, halogenated poly(vinylidene), microporous materials such as bisphenol, a microporous poly(vinylchloride), micro-porous polyamides, microporous modacrylic copolymers, microporous styrene-acrylic and its copolymers, microcrystalline cellulose, polycarbonates comprised of linear polyesters of carbonic acid in which carbonate groups reoccur in the polymer chain, porous polysulfones, polychloroethers, polyesters prepared by esterification of a dicarboxylic acid or anhydride with an alkylene polyol, poly(alkylenesulfides), phenolics, polyesters, poly(urethane), poly(imides), poly(benzimidazoles), regenerated proteins, semi-solid cross-linked poly(vinylpyrrolidone), silicon dioxide, and any combination thereof.

These and other coats utilized according to the present invention can be applied by various methods well known to the skilled artisan from other applications, including, without limitation, spray coating. Spray coating is performed using a tablet coater, fluidized bed apparatus or other suitable coating apparatus, well known to the skilled artisan for other applications.

### Enhanced Absorption Tablets

In one embodiment, the tablet is an enhanced absorption tablet. In one embodiment, the enhanced absorption tablet comprises a core comprising TH-302, or another hypoxia activated prodrug of Formula I, and one or more pharmaceutically acceptable excipients. In one embodiment, the core is surrounded by an enhanced absorption coating, which controls the release of TH-302 or other hypoxia activated prodrug of Formula I. In certain embodiments, the enhanced absorption coating consists of one coat. The advantages of the enhanced absorption tablet include lowering the amount of drug, relative to certain other types of tablets or dosage forms, required in the composition, which can lead to a reduction of side effects and/or decreased manufacturing costs.

The core of the enhanced absorption tablet comprises TH-302, or another hypoxia activated prodrug of Formula I, a binder and a lubricant, and can contain other pharmaceutically acceptable excipients. Various binders, lubricants, glidants, and other inert excipients useful in accordance with the present formulations are well known to the skilled artisan. The additional inert excipients are well known to the skilled artisan from other applications and can be found in the relevant literature, for example in the Handbook of Pharmaceutical Excipients, supra.

The enhanced absorption tablet can further comprise a coat. In one embodiment, the coat is a semi permeable coat comprising a water insoluble, water-permeable film-forming polymer, and optionally a water-soluble polymer, and yet further optionally, a plasticizer. In certain embodiments, a moisture barrier is applied directly onto the control releasing coat. In some embodiments, the moisture barrier may comprise or consist essentially of an enteric polymer (e.g. acrylic polymer), a permeation enhancer and optionally a plasticizer. Various enteric polymers, plasticizers, permeation enhancers, water insoluble, water-permeable film-forming polymers, and water-soluble polymers, useful for the extended release tablets of the invention are also useful in the enhanced absorption tablets of the invention.

### Controlled Release Matrix

In one embodiment, the formulation provided by the invention is a controlled release matrix comprising TH-302 or another hypoxia activated prodrug of Formula I. The kinetics of drug release from the matrix core depend at least in part upon the diffusion and/or erosion properties of excipients within the formulation. Suitable excipient materials for use in such controlled release matrices include, by way of example, release-resistant or controlled release materials such as hydrophobic polymers, hydrophilic polymers, lipophilic materials and mixtures thereof.

Non limiting examples of hydrophobic, or lipophilic components include glyceryl monostearate, mixtures of glyceryl monostearate and glyceryl monopalmitate, glycerylmonooleate, a mixture of mono, di and tri-glycerides, glycerylmonolaurate, paraffin, white wax, long chain carboxylic or fatty acids, long chain carboxylic acid esters, long chain fatty or carboxylic acid alcohols, and mixtures thereof. In some embodiments, the long chain carboxylic acids contain from 6 to 30 carbon atoms; in certain embodiments, at least 12 carbon atoms, and in other embodiments, from 12 to 22 carbon atoms. In some embodiments, this carbon chain is fully saturated and unbranched, while others contain one or more double bonds. In another embodiment, the long chain carboxylic acids contain 3-carbon rings or hydroxyl groups.

Non limiting examples of saturated straight chain acids include, without limitation, arachidic acid, behenic acid, caproic acid, caprylic acid, capric acid, lauric acid, montanic acid, melissic acid myristic acid, n-dodecanoic acid, n-hexadecanoic acid, n-tetradecanoic acid, palmitic acid, and stearic acid. Also useful are unsaturated monoolefinic straight chain monocarboxylic acids. Non limiting examples of these include erucic acid, gadoleic acid and oleic acid. Also useful are polyolefinic straight chain monocaboxyic acids. Non limiting examples of these include arachidonic acid, behenolic acid, linoleic acid, and linolenic acid. Useful branched acids include, for example, diacetyl tartaric acid. Non limiting examples of long chain carboxylic acid esters include glyceryl monostearates, glyceryl monopalmitates, mixtures of glyceryl monostearate and glyceryl monopalmitate, glyceryl monooleate, mixtures of glyceryl monopalmitate, glyceryl monostearate, glyceryl monooleate and glyceryl monolinoleate, glyceryl monolinolenate, glyceryl monogadoleate, mixtures of glyceryl monopalmitate, glyceryl monostearate, glyceryl monooleate, glyceryl monolinoleate, glyceryl monolinolenate and glyceryl monogadoleate, acetylated glycerides such as distilled acetylated monoglycerides, mixtures of propylene glycol monoesters, distilled monoglycerides, sodium stearoyl lactylate and silicon dioxide, mixtures of propylene glycol monoesters, distilled monoglycerides, sodium stearoyl lactylate and silicon dioxide, d-α tocopherol polyethylene glycol 1000 succinate, mixtures of mono- and diglyceride esters such as Atmul mono- and diglyceride emulsifiers, calcium stearoyl lactylate, ethoxylated mono- and di-glycerides, lactated mono- and di-glycerides, lactylate carboxylic acid ester of glycerol and propylene glycol, lactylic esters of long chain carboxylic acids, polyglycerol esters of long chain carboxylic acids, propylene glycol mono- and di-esters of long chain carboxylic acids, sodium stearoyl lactylate, sorbitan monostearate, sorbitan monooleate, other sorbitan esters of long chain carboxylic acids, succinylated monoglycerides, stearyl monoglyceryl citrate, stearyl heptanoate, cetyl esters of waxes, cetearyl octanoate, C₁₀-C₃₀ cholesterol/lavosterol esters, sucrose long chain carboxylic acid esters, and mixtures thereof.

Non limiting examples of hydrophilic polymers that can be used in certain embodiments of the controlled release matrix formulation include, without limitation, acrylic acid derivatives such as polyacrylic acid, alginic acid, carbopol, carboxymethylcellulose or other cellulose ethers, hydroxypropylmethylcellulose, hydroxypropylcellulose, hydroxyethylcellulose, polyoxyethylene, hydroxymethyl methacrylic acid polymer, polymethacrylate polymer such as, methacrylic acid polymer, hydroxyethyl methacrylic acid polymer, polyvinyl alcohols, and polyethylene oxide or polyethylene glycol.

In one embodiment, the controlled release matrix formulation further comprises one or more of a lubricant, a binder, or a plasticizer. In one embodiment, the controlled release matrix formulation further comprises one or more of a diluent, a solubilizer, a swelling enhancer, or an additive for allowing water to penetrate into the core of the formulation.

Non limiting examples of diluents include calcium hydroxyl-apatite, calcium phosphates, calcium sulfate, cellulose, cellulose derivatives, dextrin or other polysaccharides, dicalcium phosphate, dry starch, fatty acid salts such as magnesium stearate, glucose or other monosaccharides, inositol, kaolin, lactose or sucrose or other disaccharides, mannitol, sorbitol, and sucralfate.

The solubilizer can act to increase the instantaneous solubility of TH-302, or another hypoxia activated prodrug of Formula I, and can be selected from hydrophilic surfactants, lipophilic surfactants, or mixtures thereof. The surfactants can be anionic, nonionic, cationic, and zwitterionic surfactants.

The hydrophilic non ionic surfactants include, without limitation, hydrophilic transesterification products of a polyol with at least one member of the group from hydrogenated vegetable oils, and d-α-tocopheryl polyethylene glycol 1000 succinate, triglycerides, and vegetable oils, polyethylene glycol, and sorbitan fatty acid esters.

The ionic surfactants include, without limitation, acyl lactylates, alkylammonium salts, citric acid esters of mono- and di-glycerides, carnitine fatty acid ester salts, fatty acid derivatives of amino acids, fatty acid salts, fusidic acid salts, glyceride derivatives of amino acids, lecithins and hydrogenated lecithins, lysolecithins and hydrogenated lysolecithins, lysophospholipids and derivatives thereof, mono- and di-acetylated tartaric acid esters of mono- and di-glycerides, oligopeptides, polypeptides, phospholipids and derivatives thereof, salts of alkylsulfates, sodium docusate, succinylated mono- and di-glycerides, and mixtures thereof.

The lipophilic surfactants include, without limitation, acetylated glycerol fatty acid esters, fatty alcohols, glycerol fatty acid esters, hydrophobic transesterification products of a polyol with at least one member of the group from glycerides, vegetable oils, hydrogenated vegetable oils, fatty acids and sterols, lactic acid derivatives of mono- and di-glycerides, lower alcohol fatty acids esters, oil-soluble vitamins/vitamin derivatives, PEG sorbitan fatty acid esters, PEG glycerol fatty acid esters, polyglycerized fatty acid, polyoxyethylene-polyoxypropylene block copolymers, propylene glycol fatty acid esters, polyethylene glycol sorbitan fatty acid esters, polyoxyethylated sterols and sterol derivatives, polyethylene glycol alkyl ethers, sorbitan fatty acid esters, sterols and sterol derivatives, sugar esters, sugar ethers, and mixtures thereof.

In another embodiment, the solubilizers include, without limitation, betains, dioctyl sulfosuccinate, glyceryl monooleate, glycerol monolinoleate, glycerol monostearate, L-hydroxypropyl cellulose, hydroxylethylcellulose, hydroxylpropylcellulose, lauryl macrogol-32 glyceride, PEG-20-glyceryl stearate, PEG-40 hydrogenated castor oil, PEG 6 corn oil, polyglyceryl-10 mono dioleate, propylene glycol oleate, propylene glycol alginate, propylene glycol dioctanoate, propylene glycol caprylate/caprate, PEG-20 sorbitan monolaurate, PEG-4 lauryl ether, polyoxyethylene-polyoxypropylene block copolymer, polyethylene glycol 660 hydroxystearate, polyethylene glycol, sodium lauryl sulfate, sodium dodecyl sulphate, sodium taurocholate, sodium glycocholate, sodium deoxycholate, stearoyl macrogol glyceride, sucrose distearate, sucrose monopalmitate, d-α-tocopheryl polyethylene glycol 1000 succinate, and mixtures thereof. In another embodiment, the solubilizers include, without limitation, lauryl macrogol-32 glyceride stearoyl macrogol glyceride, PEG-40 hydrogenated castor oil, PEG-20 sorbitan monolaurate, PEG-4 lauryl ether, polyethylene glycol, polyoxyethylene-polyoxypropylene block copolymer, sodium lauryl sulphate, sodium dodecyl sulphate, and mixtures thereof.

Examples of swelling enhancers include, without limitation, alginates, colloidal magnesium-aluminum silicate, corn starch granules, cross-linked sodium or calcium carboxymethyl cellulose, cellulose fiber, cross-linked polyvinyl pyrrolidone, cross-linked polyacrylic acid, cross-linked Amberlite resin, low-substituted hydroxypropyl cellulose, microcrystalline cellulose, potato starch granules, pregelatinised starch, rice starch granules, sodium carboxymethyl starch and mixtures thereof.

Additives includes, without limitation, hydrophilic polymers such as polyethylene glycol (PEG), and polyvinylpyrrolidone, sugar such as D-sorbitol, xylitol, or the like, sugars such as anhydrous maltose, D-fructose, dextran (e.g. dextran 40), glucose, sucrose, or the like, surfactants such as polyoxyethylene-hydrogenated castor oil, polyoxyethylene-polyoxypropylene glycol, polyoxyethylene-sorbitan high molecular fatty acid ester, or the like, salts such as magnesium chloride, sodium chloride, or the like, organic acids such as citric acid, tartaric acid, or the like, amino acids such as β-alanine, glycine, lysine hydrochloride, or the like, and amino sugars such as meglumine.

Non limiting examples of disintegrants for use, e.g., in the matrix formulations include, without limitation, alginic acid, croscarnellose sodium, crospovidone, methacrylic acid DVB, cross-linked PVP, microcrystalline cellulose, polacrilin potassium, pregelatinized starch, sodium alginate, sodium starch glycolate, starch, and the like. In another embodiment, the disintegrants include, without limitation, cross-linked polyvinylpyrrolidone, cross-linked sodium carboxymethylcellulose, formaldehyde-casein, starch or starch derivatives such as sodium starch glycolate, or combinations with starch, swellable ion-exchange resins, such as Amberlite IRP 88, and mixtures thereof.

In another embodiment, a swellable matrix formulation is provided in which the TH-302, or another hypoxia activated prodrug of Formula I, is included in a polymeric matrix that is water-swellable rather than merely hydrophilic, and that has an erosion rate that is slower than its swelling rate, and that releases the TH-302, or another hypoxia activated prodrug of Formula I, substantially by diffusion. Non limiting examples of polymers suitable for use in the swellable matrix include, cellulose polymers and their derivatives, such as for example, carboxymethylcellulose, hydroxyethylcellulose, hydroxypropylcellulose, and microcrystalline cellulose, chitosan, crosslinked polyacrylic acids and their derivatives, maleic anhydride copolymers, polysaccharides and their derivatives, polyalkylene oxides, polyethylene glycols, poly(vinyl alcohol), poly(vinyl pyrrolidone), poly(ethyleneimine), polyurethane hydrogels, starch and starch-based polymers, poly (2-ethyl-2-oxazoline), xanthan gum, and mixtures thereof.

Methods of manufacturing controlled release matrices, which are well known to the skilled artisan, include wet granulation, dry granulation (e.g. slugging, roller compaction), direct compression, melt granulation, melt extrusion, and rotary granulation. Controlled release particles utilized in controlled release matrixes, which can be compressed or placed in capsules, can be produced by combining TH-302, or another hypoxia activated prodrug of Formula I, and a hydrophobic fusible component and/or a diluent. Controlled release matrices can also be produced by mechanically working a mixture of TH-302, or another hypoxia activated prodrug of Formula I, a hydrophobic fusible component, and optionally a release component including a water soluble fusible material or a particulate material under mixing conditions that yield aglomerates, breaking down the agglomerates to produce controlled release seeds having desired release properties, and optionally adding more carrier or diluent and repeating the mixing steps until controlled release seeds having desired release properties are obtained. These particles also can be size separated (e.g. by sieving) and compressed into a matrix, or even encapsulated in capsules.

### Microparticles

In one embodiment, the formulation of TH-302, or another hypoxia activated prodrug of Formula I, is a multiparticulate system, which contains multiple microparticles containing TH-302 or another hypoxia activated prodrug of Formula I, and one or more pharmaceutically acceptable excipients. The microparticles can be contained within a capsule or can be compressed into a matrix or tablet that upon ingestion dissolves into multiple sub-units, wherein the sub-units or pellets possess the desired controlled release properties of the formulation. The multiparticulates or the multiple unit formulations can be surrounded by one or more coatings. Examples of such coatings include, without limitation, polymeric controlled release coatings, delayed release coatings, enteric coatings, immediate release coatings, taste-masking coatings, extended release coatings, and non functional coatings. The excipient includes, without limitation, one or more of anti-foaming agents, binders, chemical stabilizers, coloring agents, diluents, disintegrating agents, emulsifying agents, fillers, flavoring agents, glidants, lubricants, pH modifiers, spheronization aids, solubility enhancers, and suspending agents.

Solubility enhancers can be any surfactant suitable for use in pharmaceutical compositions, which can be anionic, cationic, zwitterionic or non ionic surfactants. The microparticles provided herein can be further modified by being coated with a control releasing coat.

Microparticles of TH-302 or another hypoxia activated prodrug of Formula I can be prepared by a number of different procedures, such as spray drying, fluidized bed based granulation/pelletization process, a spheronization process, and the like, which are well known to the skilled artisan.

### Drug Layered Particles

In one embodiment, the microparticle formulation comprises layered microparticles. Such layered microparticles can be made by coating a particle or core, such as a sugar sphere, with TH-302 or another hypoxia activated prodrug of Formula I, and optionally a polymeric binder. In various embodiments, the particle or core either contains TH-302 or another hypoxia activated prodrug of Formula I or is inert (does not contain any prodrug). In certain embodiments, the inert cores include or are composed of water-insoluble materials such as cellulose spheres or silicon dioxide. In other embodiments, the inert cores include or are composed of water-soluble materials such as starch, salt or sugar spheres.

### Nanoparticles

In one embodiment, the formulation of TH302 or another hypoxia activated prodrug of Formula I comprises a nanoparticle. Nanoparticles as used herein are submicron (e.g, and without limitation, <1 μm) particles, such as colloidal particles. This includes nanoparticles or nanospheres in which the drug is adsorbed, dissolved, or otherwise included throughout the particle or sphere, and nanocapsules in which the drug is confined to an aqueous or oily core surrounded by a shell-like wall.

Nanoparticles can be made from one or more of biocompatible and biodegradable materials such as polymers, either natural (e.g., gelatin, albumin) or synthetic (e.g., polylactides, polyalkylcyanoacrylates), or solid lipids. In the body, the drug (prodrug) is usually released from the nanoparticle by one or more of diffusion, swelling, erosion, or degradation. Nanoparticles useful in the invention also include those that provide for controlled and/or sustained drug release from the nanoparticle. Using the information provided herein and existing technologies, one of skill in the art can prepare nanoparticles suitable for drug formulation. See, for example, Bala et al. PLGA Nanoparticles In Drug Delivery: The State Of The Art, Crit. Rev. Ther. Drug Carrier Syst. 2004, 21: 387-422; Vauthier et al., Poly(alkylcyanoacrylates) As Biodegradable Materials For Biomedical Applications, Adv. Drug Deliv. Rev. 2003, 55: 519-548; Couvreur et al., Nanocapsule Technology: A Review, Crit. Rev. Ther. Drug Carrier Syst., 2002, 19: 99-134, each of which is incorporated herein by reference.

### Pulsatile Release Formulation

In one embodiment, the formulation of the present invention provides for pulsatile release of TH-302 or another hypoxia activated prodrug of Formula I. Pulsatile release refers to drug release at one or more time intervals, for example at an initial quick release followed by a slow release of the drug or an initial quick release followed by, after some period of time, usually 1 to 4 hours, another quick release. See, for example, U.S. Pat. Nos. 5,011,692 and 5,980,508, each of which is incorporated herein by reference. For example, and without limitation, by combining uncoated, taste-masked or enteric coated microparticles with delayed or sustained release coated microparticles, a pulsatile drug release profile or chronotherapeutic profile can be achieved. For example, and without limitation, an excipient suitable for pulsatile release formulations of the present invention include the collagen, atelocollagen.

### Unit Dose Formulations and Treatment Methods

In one embodiment, the present invention provides unit dose forms of the oral formulations of TH-302 or another hypoxia activated prodrug of Formula I provided herein. In various embodiments, the unit dose form contains about 25 mg-about 1000 mg, about 50 mg-about 900 mg, about 75 mg-about 700 mg, about 100 mg-about 600 mg, about 25 mg-about 100 mg, or about 700 mg-about 1000 mg of TH-302 or another hypoxia activated prodrug of Formula I.

Unit dose forms of the invention containing about 50 mg, about 75 mg, about 100 mg, about 150 mg, about 200 mg, or about 250 mg of TH-302 or another hypoxia activated prodrug of Formula I formulated for immediate release are especially useful, in that such unit doses can be conveniently administered QD, BID, or TID, and, depending on the BSA and dosing frequency, one or more than one such unit dose can be administered per dose. In various embodiments, the unit dose forms are brightly colored or strikingly patterned or both (for example, black and yellow striped tablets or capsules) to permit easy identification or reduce the likelihood of confusion with other medications.

In another embodiment, the present invention provides a method of treating cancer comprising administering a therapeutically effective amount of a formulation provided herein to a patient in need of such treatment. In one embodiment, TH-302 or another hypoxia activated prodrug of Formula I is administered as the sole anti cancer agent (monotherapy). In one embodiment, TH-302 or another hypoxia activated prodrug of Formula I is administered in combination with another anti cancer agent (combination therapy) or anti cancer therapy. Methods of treating cancer by administering TH-302 or other hypoxia activated prodrugs of Formula I, therapeutically effective amounts of prodrug utilized for such treatments, the frequency of administration, and the sequence of administration when administered in combination with another anti cancer agent, and such other anti cancer agents suitable for coadministration are disclosed in PCT Pub. Nos. WO 2007/002931; WO 2008/083101; WO 2010/048330; WO 2012/006032; and WO 2012/009288; and PCT patent application No. PCT/US2012/031677, each of which is incorporated herein by reference, and can be adapted in accordance with the present disclosure for practicing the present treatment methods.

In one embodiment, the patient's cancer treated is a metastatic cancer or a refractory and/or relapsed cancer that is refractory to first, second, or third line treatment. In another embodiment, the treatment is a first, a second, or a third line treatment. As used herein, the phrase “first line” or “second line” or “third line” refers to the order of treatment received by a patient. First line treatment regimens are treatments given first, whereas second or third line treatment are given after the first line therapy or after the second line treatment, respectively. Therefore, first line treatment is the first treatment for a disease or condition. In patients with cancer, primary treatment can be surgery, chemotherapy, radiation therapy, or a combination of these therapies. First line treatment is also referred to those skilled in the art as primary therapy or primary treatment. Typically, a patient is given a subsequent chemotherapy regimen because the patient did not show a positive clinical or only showed a sub-clinical response to the first line therapy, or the first line treatment has stopped.

In various embodiments, the cancer treated is selected from the group consisting of anal cancer, breast cancer, esophageal cancer, lung cancer (both small cell and non-small cell), melanoma, ovarian cancer, pancreatic cancer, prostate cancer, sarcoma, soft tissue sarcoma, and any solid tumor cancer. In various other embodiments, the cancer treated is selected from a blood cancer, non limiting examples of which include various leukemias. Treatment of hyperproliferative diseases other than cancer is also contemplated according to the present invention.

The invention being described in summary and in detail, is illustrated and not limited by the examples below.

### EXAMPLES

### Example 1

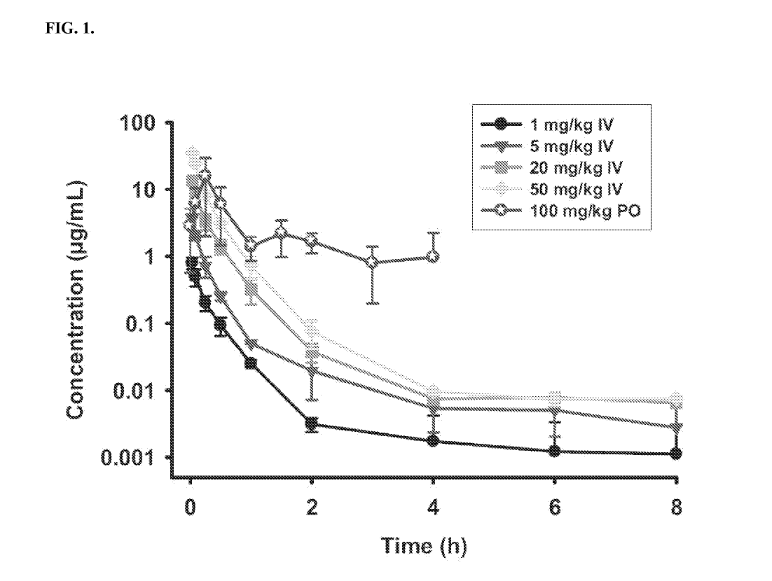

This example demonstrates TH-302's surprisingly high oral bioavailability. TH-302 (1-methyl-2-nitro-1H-imidazole-5-yl) N,N′-bis(2-bromoethyl) diamidophosphate, m.w.=449) that was about 96% pure based on high performance liquid chromatography (HPLC) analysis, was used. Male Sprague-Dawley rats (n=3 per time point) were administered a single oral dose of 100 mg/kg TH-302 (see, Table 1 and FIG. 1). TH-302 was rapidly and well absorbed with mean peak plasma concentration reached at the first sampled time point of 15 minutes and an absolute oral bioavailability of 77.3%. The corresponding mean area under the curve (AUC) and half-life were 13.1 μg-h/mL and 1.6 h, respectively. Following the same dose of 100 mg/kg by a 30 minute IV infusion (Table 2), the mean peak plasma concentration at the end of infusion was 30.9 μg/mL with a mean AUC and half-life of 17.1 μg-h/mL and 0.27 h, respectively. This example demonstrates that TH-302 and such other drugs are suitable for administration as an oral formulation.

TABLE 1
Pharmacokinetics of TH-302 in rats (mean ± standard deviation (S.D.))

| | | Paramet | | | | |
|---|---|---|---|---|---|---|
| | | Rat (n = 3) | | | | |
| | | Route | | | | |
| | Unit | IV | IV^{b} | IV | IV | PO^{a} |
| Dose | mg/kg | 1 | 5 | 20 | 50 | 100 |
| Tₘₐₓ | h | 0.033 ± 0.00 | 0.033 ± 0.00 | 0.033 ± 0.00 | 0.033 ± 0.00 | 0.25 |
| Cₘₐₓ | μg/m | 0.802 ± 0.165 | 3.82± | 13.5 ± 1.52 | 35.5 ± 3.01 | 15.8 |
| AUC | μg- | 0.221 ± 0.054 | 0.826± | 3.30 ± 0.36 | 8.47 ± 0.02 | 13.1 |
| Cl | L/h/kg | 4.69 ± 1.05 | 6.14 ± 0.99 | 6.09 ± 0.68 | 5.91 ± 0.25 | — |
| Vₛₛ | L/kg | 2.05 ± 0.69 | 5.09 ± 3.66 | 2.29 ± 0.61 | 1.67 ± 0.04 | — |
| t_{1/2} | h | 2.03 ± 0.24 | 3.75 ± 3.58 | 4.37 ± 0.42 | 1.32 ± 0.80 | 1.59 |
| Oral F | % | — | — | — | — | 77.3 |

^{a}Composite data, n = 3 per time point
^{b}n = 2
AUC: total area under the concentration-time curve from zero to infinity;
IV: intravenous;
PO: oral

TABLE 2
Single dose pharmacokinetic parameters of TH-302 after
a 30-min intravenous infusion of TH-302 in rats^{a}

| | | | | AUC | Half- | | |
|---|---|---|---|---|---|---|---|
| | Dose | Tₘₐₓ | Cₘₐₓ | (μg- | Life | Cl | Vₛₛ |
| Gender | (mg/kg) | (h) | (μg/mL) | h/mL) | (h) | (L/h/kg) | (L/kg) |
| F | 50 | 0.517 | 14.1 | 7.33 | 0.319 | 6.87 | 2.81 |
| | 100 | 0.500 | 30.0 | 16.1 | 0.286 | 6.16 | 2.59 |
| | 200 | 0.683 | 71.1 | 40.6 | 0.390 | 4.84 | 2.35 |
| M | 50 | 0.517 | 15.6 | 7.94 | 0.333 | 6.27 | 2.57 |
| | 100 | 0.550 | 30.9 | 17.0 | 0.268 | 5.90 | 2.59 |
| | 200 | 0.550 | 62.5 | 37.6 | 0.323 | 5.38 | 2.51 |

^{a}n = 3 rats per time point
^{b}n = L

Example 2 below provides immediate release oral formulations of the invention. As used herein, an “active agent” refers to TH-302 or another compound of Formula I.

### Example 2

A crystalline form of the active agent (about 100 mg-about 500 mg) is encased in gelatin capsules to provide an immediate release formulation of the active agent in various unit dose forms. Alternatively, microparticle formulations containing about 100 mg-about 500 mg of the active agent, and lactose, sucrose, or another sugar, are encased in gelatin capsules to provide a rapid release formulation of the active agent in various unit dose forms.

Examples 3-7 below provide modified release oral formulations of an active agent.

### Example 3

About 500 g of the active agent, about 600 g of poly(ethylene oxide) possessing about a 100,000 molecular weight, and about 50 g of polyvinylpyrrolidone having an average molecular weight of about 40,000 are added to a mixing bowl and the ingredients dry mixed for 10 minutes. Then, about 300 g of anhydrous ethanol is added slowly with continuous blending for 10 minutes to prepare a granulation mixture. The freshly prepared granulation mixture is passed through a 20 mesh screen, allowed to dry at 25° C. for about 20 hours, and then passed through a 16 mesh screen. Next, the granulation mixture is transferred to a mixer, and lubricated with about 10 g of magnesium stearate to produce an oral formulation of the present invention. The formulation is compressed, under pressure, into tablets comprising, for example, about 100 mg to about 500 mg of the active agent.

### Example 4

Oral formulations of the present invention are manufactured by following the procedure of Example 3, to provide unit dose forms comprising (i) from about 50 mg to about 500 mg of an active agent; (ii) from about 10 mg to about 500 mg of a poly(alkylene oxide) polymer of about 100,000 to about 500,000 molecular weight, e.g., poly(ethylene oxide), poly(propylene oxide), poly(isopropylene oxide), and poly(butylene oxide); or from about 10 mg to about 500 mg of a polymer of about 7,500 to 325,000 molecular weight selected from an alkali carboxymethylcellulose and an alkaline earth carboxymethylcellulose; (iii) about 0.5 mg to about 50 mg of a poly(vinyl) polymer from about 5,000 to about 300,000 molecular weight, e.g., polyvinylpyrrolidone; copolymers of vinyl pyrrolidone and one or more of vinyl acetate, vinyl chloride, vinyl fluoride, vinyl butyrate, vinyl laurate, and vinyl stearate; and (iv) up to about 7.5 mg of a lubricant selected from polyethylene glycol, magnesium stearate, calcium stearate, potassium oleate, sodium stearate, stearic acid, and sodium palmitate. Other oral formulations of the present invention may contain other excipients, for example, colorants, compression aids such as microcrystalline cellulose and the like, and binders such as starch and the like. The formulation is compressed at a ⅛ to 3 ton-force to yield an orally administrable tablet.

### Example 5

The tablets of Examples 3 and 4 are coated to provide unit dose forms of the invention as follows. First, a coating solution, which can be hydrophilic or hydrophobic, is prepared. Ethyl cellulose (about 154 g) having a molecular weight of about 220,000 and an ethoxyl content of about 50 weight %, about 112 g of hydroxypropylcellulose of about 80,000 molecular weight and a molar substitution of 3, and about 15 g of polyoxyethylene (40) stearate are dissolved with stirring in about 3,700 g of anhydrous ethanol. The solution resulting is allowed to stand without stirring for 3 days to provide coating composition-1.

Another type of coating solution is prepared by dissolving about 160 g of cellulose acetate having an acetyl content of about 40 weight % and a molecular weight of about 40,000, and about 90 g of ethylene oxide-propylene oxide-ethylene oxide triblock copolymer of about 8,400 molecular weight and an ethylene oxide content of about 80 wt % in about 4,700 g of anhydrous acetone with stirring and slight warming, if required, to 26° C. The resulting mixture is allowed to stand at ambient room temperature for one day to provide coating composition-2.

Next, the active agent-tablets are placed into a pan coater. The coating compositions are sprayed onto the tablets in a current of warm air until a coat with a desired thickness is applied to the tablets. The coated tablets are dried in a forced air oven, e.g., at 40° C. for 24 hrs or at a lower temperature depending on the stability of the formulated active agent.

### Example 6

A modified release microparticle formulation capable of slowly releasing the active agent over a period time is prepared as follows. Microcrystalline cellulose (about 160 g), lactose (about 75 g), citric acid (up to about 40 g), and an active agent (about 100 g) is mixed and kneaded using water in a planetary mixer to form a wet mass. The wet mass is passed through a Nica E140 extruder to form an extrudate (about 1 mm diameter). The extrudate is then passed through a Nica spheronizer to form microparticles, which are then dried in a tray drying oven or in a fluid bed dryer to provide microparticle formulations of the present invention. These are filled into hard shell pharmaceutical capsules, or coated, or layered, as described herein to provide other oral formulations of the present invention.

### Example 7

In this example, microparticles prepared as above are coated via a two step process as follows. An aqueous solution of hydroxypropyl methyl cellulose (about 7.5% by weight) and polyethylene glycol (about 2.5% by weight) is prepared and sprayed on to the microparticles to form a seal coat. The seal coated microparticles are then coated with a barrier coat using a commercially available aqueous dispersion of ethyl cellulose (e.g., Aquacoat® 30% by weight) mixed with acetylated monoglycerides (about 9.5% by weight). The coated microparticles may be filled into hard shell pharmaceutical capsules, or layered as described herein to provide other oral formulations of the present invention.

Example 8 below describes oral formulations of the present invention that provide pulsatile release of the active agent.

### Example 8

A mixture of about 15 mg of atelocollagen and 1 mg of an active agent is compression-molded on a tableting machine (400 kg/cm²) and 35 mg of atelocollagen is again compression-molded thereon. Thus is produced an active agent containing collagen layer and an active agent free collagen layer. The above procedure is repeated three more times, so that four layers of each layer are formed. A cylindrical pellet having a thickness of about 1.5 mm, a diameter of about 10 mm and a weight of about 200 mg is thus produced. Separately, about 10 g of Silastic® 382 silicone base and about 2 drops of stannous octoate are mixed together quickly. The mixture is placed in a vessel with a diameter of 15 mm and a depth of 5 mm. The above laminated cylindrical pellet is immersed in said mixture with the top (an active agent-containing collagen layer) being left in contact with air. The silicone polymer is cured by allowing the mixture with the pellet immersed therein to stand at room temperature for 24 hours. Thereafter the formulation is taken out of the vessel. The dissolution of the active agent from the coated pharmaceutical preparation thus obtained is determined using physiological saline at room temperature and sampling the saline at timed intervals for determining the quantity of active agent released within a unit time by high-performance liquid chromatography.

Microparticles of the active agent as exemplified above, or those containing an immediate release core and optionally an immediate release coat, can be embedded in atelocollagen as described in this example, to prepare pulsatile release oral formulations of the present invention.

It should be understood that although the present invention has been specifically disclosed by certain aspects, embodiments, and optional features, modification, improvement and variation of such aspects, embodiments, and optional features can be resorted to by those skilled in the art, and that such modifications, improvements and variations are considered to be within the scope of this disclosure.

The inventions have been described broadly and generically herein. Each of the narrower species and subgeneric groupings falling within the generic disclosure also form part of the invention. In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

1. An oral formulation comprising a hypoxia activated prodrug of Formula I, 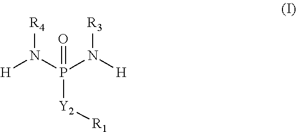
wherein
Y₂ is O, S, NR₆, NCOR₆, or NSO₂R₆
R₆ is C₁-C₆ alkyl, C₁-C₆ heteroalkyl, aryl, or heteroaryl;
R₃ and R₄ are independently selected from the group consisting of 2-haloalkyl, 2-alkylsulfonyloxyalkyl, 2-heteroalkylsulfonyloxyalkyl, 2-arylsulfonyloxyalkyl, and 2-heteroalkylsulfonyloxyalkyl;
R₁ has the formula L-Z₃;
L is C(Z₁)₂;
each Z₁ independently is hydrogen, halogen, C₁-C₆ alkyl, C₁-C₆ heteroalkyl, aryl, heteroaryl, C₃-C₈ cycloalkyl, heterocyclyl, C₁-C₆ acyl, C₁-C₆ heteroacyl, aroyl, or heteroaroyl;
or L is: 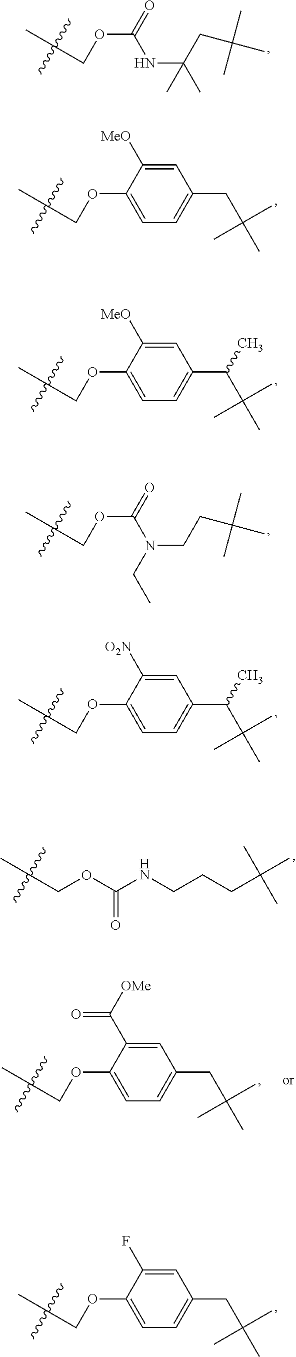
Z₃ is a bioreductive group having a formula selected from the group consisting of: 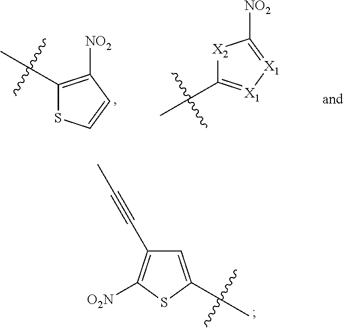
each X₁ is independently N or CR₈;
X₂ is NR₇, S, or O;
each R₇ is independently C₁-C₆ alkyl, C₁-C₆ heteroalkyl, C₃-C₃ cycloalkyl, heterocyclyl, aryl or heteroaryl;
and R₈ is independently hydrogen, halogen, cyano, CHF₂, CF₃, CO₂H, amino, C₁-C₆ alkyl, C₁-C₆ heteroalkyl, C₁-C₆ cycloalkyl, C₁-C₆ alkoxy, C₁-C₆ alkylamino, C₁-C₆ dialkylamino, aryl, CON(R₇)₂, C₁-C₆ acyl, C₁-C₆ heteroacyl, aroyl or heteroaroyl;
or a pharmaceutically acceptable salt thereof;
wherein the oral formulation is a modified release formulation.

2. The modified release formulation of claim 1 that is a tablet.

3. The modified release formulation of claim 1 that comprises microparticles.

4. The modified release formulation of claim 1 that comprises a controlled release matrix.

5. The modified release formulation of claim 1, further comprising a core.

6. The modified release formulation of claim 1, further comprising a coat.

7. The modified release formulation of claim 6, wherein the coat is a controlled release coat.

8. The modified release formulation of claim 6, wherein the coat is a moisture barrier coat.

9. The modified release formulation of claim 1 that comprises nanoparticles.

10. The modified release formulation of claim 1, further comprising one or more of an additive, an anti-foaming agent, a binder, a chemical stabilizer, a coloring agent, a diluent, a disintegrating agents, an emulsifying agent, a filler, a flavoring agents, a glidant, a lubricant, a pH modifier, a plasticizer, a solubilizer, a swelling enhancer, a spheronization aid, a solubility enhancer, and a suspending agent.

11. The modified release formulation of claim 1 that shows a pulsatile release profile of the hypoxia activated prodrug.

12. An oral, immediate release, microparticulate or nanoparticulate oral formulation comprising a hypoxia activated prodrug of Formula I 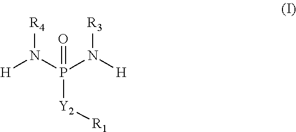
wherein
Y₂ is O, S, NR₆, NCOR₆, or NSO₂R₆
R₆ is C₁-C₆ alkyl, C₁-C₆ heteroalkyl, aryl, or heteroaryl;
R₃ and R₄ are independently selected from the group consisting of 2-haloalkyl, 2-alkylsulfonyloxyalkyl, 2-heteroalkylsulfonyloxyalkyl, 2-arylsulfonyloxyalkyl, and 2-heteroalkylsulfonyloxyalkyl;
R₁ has the formula L-Z₃;
L is C(Z₁)₂;
each Z₁ independently is hydrogen, halogen, C₁-C₆ alkyl, C₁-C₆ heteroalkyl, aryl, heteroaryl, C₃-C₈ cycloalkyl, heterocyclyl, C₁-C₆ acyl, C₁-C₆ heteroacyl, aroyl, or heteroaroyl;
or L is: 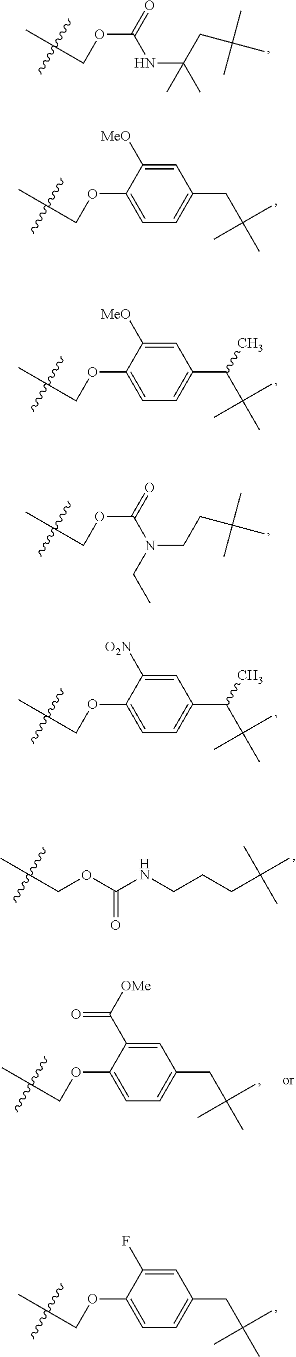
Z₃ is a bioreductive group having a formula selected from the group consisting of: 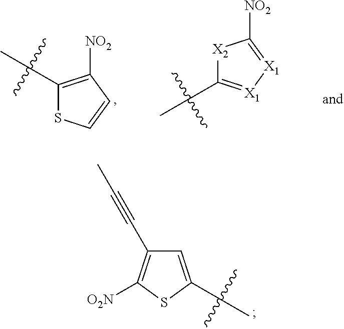
each X₁ is independently N or CR₈;
X₂ is NR₇, S, or O;
each R₇ is independently C₁-C₆ alkyl, C₁-C₆ heteroalkyl, C₃-C₈ cycloalkyl, heterocyclyl, aryl or heteroaryl;
and R₈ is independently hydrogen, halogen, cyano, CHF₂, CF₃, CO₂H, amino, C₁-C₆ alkyl, C₁-C₆ heteroalkyl, C₁-C₆ cycloalkyl, C₁-C₆ alkoxy, C₁-C₆ alkylamino, C₁-C₆ dialkylamino, aryl, CON(R₇)₂, C₁-C₆ acyl, C₁-C₆ heteroacyl, aroyl or heteroaroyl;
or a pharmaceutically acceptable salt thereof;
and one or more pharmaceutically acceptable excipients.

13. A gelatin capsule or a tablet formulation comprising the immediate release formulation of claim 12.

14. The oral formulation of claim 1, wherein the hypoxia activated prodrug is TH-302.

15. A unit dose of the oral formulation of claim 14.

16. The unit dose of claim 15 that comprises about 25 mg-about 1000 mg TH-302.

17. The unit dose of claim 16 that comprises about 50 mg-about 500 mg TH-302.

18. The unit dose of claim 17 that comprises about 75 mg TH-302.

19. A method of treating cancer comprising administering a therapeutically effective amount of the formulation of claim 14 to a patient in need of such treatment.

20. The oral formulation of claim 12, wherein the hypoxia activated prodrug is TH-302.

21. A unit dose of the oral formulation of claim 20.

22. The unit dose of claim 21 that comprises about 25 mg-about 1000 mg TH-302.

23. The unit dose of claim 22 that comprises about 50 mg-about 500 mg TH-302.

24. The unit dose of claim 23 that comprises about 75 mg TH-302.

25. A method of treating cancer comprising administering a therapeutically effective amount of the formulation of claim 20 to a patient in need of such treatment.
